(19) United States
(12) Patent Application Publication
(10) Pub. No.: US 2008/0108054 A1
(43) Pub. Date: May 8, 2008

(54) Detecting early HIV infection in genital tract cells and secretions

(76) Inventors: Jayasri Basu, Bronx, NY (US); Seymour L. Romney, White Plains, NY (US); Ruth Hogue Angeletti, New Rochelle, NY (US)

Correspondence Address: AMSTER, ROTHSTEIN & EBENSTEIN LLP, 90 PARK AVENUE, NEW YORK, NY, 10016, US

(21) Appl. No.: 11/895,686

(22) Filed: Aug. 27, 2007

Related U.S. Application Data
(60) Provisional application No. 60/840,781, filed on Aug. 28, 2006.

(51) Int. Cl. C12Q 1/70

(52) U.S. Cl. 435/5

(57) ABSTRACT

Provided are methods of diagnosing HIV in a woman. Also provided are methods of diagnosing HIV in an HIV-seronegative woman. Additionally provided are methods of determining whether to recommend that a woman should undergo anti-HIV therapy. The methods comprise detecting HIV in a sample of genital tract cells and/or genital tract secretions from the woman.

### CROSS-REFERENCE TO RELATED APPLICATION

This application claims the benefit of U.S. Provisional Application No. 60/840,781, filed Aug. 28, 2006.

### BACKGROUND OF THE INVENTION

(1) Field of the Invention

The present invention generally relates to methods for detecting HIV. More specifically, the invention provides methods of detecting HIV infection in cells and secretions of the female genital tract.

(2) Description of the Related Art

HIV testing is integral to HIV prevention, treatment and care efforts. Knowledge of one's HIV status is critical for preventing the spread of disease. Hence, early diagnosis of HIV infection is always preferred. Screening provides an opportunity for people to receive counseling and information about risk reduction. Early knowledge of HIV status, particularly for those who are serologically HIV positive, can link them to medical care and services that can reduce morbidity and mortality and improve their quality of life.

Methods of HIV testing available in the United States differ based on the type of specimen tested (e.g., whole blood, serum, plasma, oral fluid, urine) and how quickly the results are available (conventional or rapid). Detection of HIV antibodies in the blood continues to be the gold standard. The drawback of this method is that the appearance of HIV antibodies in the blood of an HIV-exposed individual is entirely dependent on the host's immune system and viral characteristics. Unless the host's immune system is challenged enough by the virus, a humoral immune response is not initiated. A lag phase elapses between HIV exposure and initiation of HIV antibody response, the duration of which varies from individual to individual. If we were to rely on the appearance of HIV antibodies in the blood to determine an individual's HIV status, this valuable window of time that could be utilized to prevent the virus from targeting the immune system not only remains unavailable, but more importantly remains unutilized. The modes transmission of HIV has changed from the past. In the 1980s, needle sharing, blood transfusion or organ donations were the primary modes of HIV transmission. In recent times, heterosexual transmission accounts for 90% of new HIV infection worldwide; and the semen, vaginal secretions and breast milk of HIV infected individuals are the primary modes of transmission of HIV today. Therefore, HIV antibody screening of the blood may no longer fulfill the criterion of being the most effective strategy and method in determining the HIV status of an individual.

Cervical cancer mortality in the United States has declined by more than 70% since 1950 due in large part to early detection with Papanicolaou (Pap) test (American Cancer Society, 2002). Cervical cancer is still a huge burden for women in developing nations where Pap smear screening remains unavailable (Suba et al., 2006). In 1993, the US Center for Disease Control and Prevention added invasive cervical cancer to the list of acquired immunodeficiency syndrome (AIDS)-defining illnesses (Phelps et al., 2001). The association between immunosuppression and development of cervical intraepithelial neoplasia (CIN) occurs irrespective of the source of immunosuppression, whether congenital, iatrogenic, or acquired (Porreco et al., 1975). In Human Immunodeficiency Virus (HIV) infected women, the CIN lesions are often multifocal, progress rapidly, have high recurrence rates and require more stringent monitoring and intervention (Clarke and Chetty, 2002).

Pap smear or genital tract cell and secretion collection methods such as cervicovaginal lavage (CVL), offer clinicians and investigators a simple noninvasive method of sampling cells and mucus secreted by the uterus, cervix and vaginal tissues in a state readily amenable to in vitro studies. CVL sampling is a routine gynecological procedure found to be especially convenient for maximum sensitivity in the detection of Human Papillomavirus (HPV) infection (Burk et al., 1986; Vermund et al. 1989).

It would be desirable to further determine whether Pap smears and other samples of cervical cells or secretions can be analyzed to determine whether a woman has an HIV infection. The present invention addresses that need.

### SUMMARY OF THE INVENTION

Accordingly, the inventors have discovered that HIV can be detected in genital tract cells or secretions, from patients who are persistently HIV seronegative.

The invention is directed to methods of diagnosing HIV in a woman. The methods comprise obtaining a sample of genital tract cells and/or genital tract secretions from the woman; evaluating the sample for the presence of HIV; and determining whether the woman is HIV positive or HIV negative from the sample. The presence of HIV in the sample indicates the woman is HIV positive, and the absence of HIV in the sample indicates the woman is HIV negative.

The invention is also directed to methods of diagnosing HIV in an HIV-seronegative woman. The methods comprise obtaining a sample of genital tract cells and/or genital tract secretions from the woman; evaluating the sample for the presence of HIV; and determining whether the woman is HIV positive or HIV negative from the sample. The presence of HIV in the sample indicates the woman is HIV positive, and the absence of HIV in the sample indicates the woman is HIV negative.

Additionally, the invention is directed to methods of determining whether to recommend that a woman should undergo anti-HIV therapy. The methods comprise obtaining a sample of genital tract cells and/or genital tract secretions from the woman; and evaluating the sample for the presence of HIV. The presence of HIV in the sample indicates that anti-HIV therapy should be recommended to the woman.

### BRIEF DESCRIPTION OF THE DRAWINGS

FIG. 1 is a representation of the protein profiles of cervicovaginal lavage samples after Coomassie staining following sodium dodecyl sulfate polyacrylamide gel electrophoresis (SDS-PAGE). Protein concentrations of the cervicovaginal lavage (CVL) samples ranged from 0.18-1.34 mg/ml. 20 μl CVL sample was loaded per lane. The Coomassie stained gel patterns of 20 individual CVL samples show distinct polypeptide bands across a wide range of molecular weights, indicating that detectable proteins can be retrieved in a CVL sample.

FIG. 2 represents the search results from the NCBI Protein Data Bank when trypsin digested peptides obtained during mass spectrometry processing were submitted for protein identifications. Panel C shows that the Protein Data Bank identified HIV-1 env glycoprotein to be present in the CVL sample analyzed. Panel A represents a typical spectrum obtained during mass spectrometry, Panel B shows the parameters submitted to the NCBI Protein Data Bank, Panel C shows the lists of the candidate proteins identified by the Protein Data Bank to be present in the CVL sample, and Panel D shows a peptide coverage map substantiating that HIV-1 env glycoprotein is indeed present in the sample.

FIG. 3 shows search results received from the NCBI Protein Data Bank identifying the HIV-1 gag protein to be present in a the CVL sample analyzed

FIG. 4 shows the protein profiles of CVL samples and the western blot data that identifies HIV viral proteins, p24 and gp41 to be present in some of the CVL samples, and diagrams of portions of the HIV genome. Panel A shows a blot with seven CVL samples run in duplicates. One half of each gel was stained with Coomassie brilliant blue which exhibits the protein profiles of the samples. The proteins on the other half were immunoblotted onto nitrocellulose membranes and then probed with p24 MAB (dilution 1:500). Gel 1 depicts four MALDI TOF HIV-1 positive CVL samples that are also HIV-1 p 24 positive by WB. Lanes 2, 4, 5 of Gel 2 shows HIV-1 p 24 negative CVL samples. Lanes 3 of Gel 1 and Gel 2 represent the same CVL sample. Panel B shows gels and blots where four CVL samples were run on a gel in duplicates. One half of the gel was stained with Coomassie stain and the proteins of the other half was transferred on to a nitrocellulose membrane and the blot was probed with HIV-1 gp41 MAB (dilution 1:500). The WB data as shown in the bottom panel identifies gp41 positive CVL samples in lanes 3 and 4 and a gp41 negative CVL sample in lane 2.

FIG. 5 represents western blot data showing that immunoglobulins inherent in the CVL samples are not responsible for HIV-1 antigen positive bands. Four CVL samples were run on a gel in duplicates. The proteins were transferred onto a nitrocellulose membrane. The immunoblot was then divided into two. One half was probed with HIV-1 p24 MAB (1:500), followed with HRP-conjugated IgG (1:3000). The other half was probed with HRP conjugated IgG alone (1:3000). Both halves were then treated with a chemiluminscent reagent to visualize the protein. In the left panel, lane 1 shows an HIV-1 p24 negative CVL sample and lanes 2-4 show three HIV-1 p24 positive CVL samples. The right panel shows the half of the same gel that was processed simultaneously without the primary antibody. The absence of immunopositive bands in the gel shows that the immunoglobulin inherent in the CVL samples are not responsible for the immunopositive bands.

FIG. 6 shows the western blots data showing that the HIV-1 p24 antigen in the CVL is phosphorylated. The immunoaffinity column chromatography procedure performed prior to the WB, is described under “western blot assays, section (c)” in the Methods section of Example 1. Lane 1—Molecular weight marker; Lane 2-HIV-1 p24 full length recombinant protein (Positive control); Lane 3—CVL sample; Lane 4—Protein A+Protein G treated CVL sample; Lane 5—Experimental immunoaffinity column eluate; Lane 6—Control immunoaffinity column eluate. Panels a and b show the immunoblots probed with HIV-1 p24 MAB and anti-phosphotyrosine recombinant 4G10 MAB, respectively. Collectively the experiments presented in FIG. 6 confirm that (a) the CVL sample was positive for HIV-1 p24, (b) HIV-1 p24 protein in the CVL sample was phosphorylated and, (c) the immunoglobulins in CVL samples were not responsible for the immunoreactive bands.

FIG. 7 is a photograph of a western blot showing the absence of prostate specific antigen (PSA) in HIV-1 p24 antigen positive CVL samples. Several HIV p24 confirmed CVL samples along with HIV p24 negative CVL samples were run on a gel. After transferring the proteins, the immunoblot was first probed with prostate specific antigen MAB (1:500), then with HRP-conjugated IgG, and immunoreactive bands were visualized with an enhanced chemiluminescent reagent. The HIV p24 confirmed and HIV negative CVL samples are labeled (+) and (−), respectively. Lanes 4 and 5 show the absence of PSA in HIV-1 p24 positive CVL samples.

FIG. 8 is a photograph of a western blot showing that the 41 kDa protein in the CVL is an HIV-1 antigen. Four equal aliquots of a CVL sample were run on a gel. The proteins were transferred to a nitrocellulose membrane. The immunoblot was split into 4 sections. Each section was then probed with HIV-1 p24 monoclonal antibody (1:500) obtained from four different sources, followed with HRP conjugated IgG (1:3000). The immunoreactive bands were visualized using an enhanced chemiluminescent reagent.

1 denotes the immunoblot probed with HIV-1 p24 MAB obtained from Abcam.

2 denotes the immunoblot probed with HIV-1 p24 MAB obtained from Cliniqa

3 denotes the immunoblot probed with HIV-1 p24 MAB obtained from Zeptometrix

4 denotes the immunoblot probed with HIV-1 p24 MAB obtained from New York Blood Center courtesy of Dr. Shibo Jian. The identical data obtained in the WB results when each of the different monoclonal antibodies were used, demonstrate that HIV-1 p24 was indeed present in the CVL sample.

FIG. 9 represents the micrographs showing immunohistochemical localization of HIV p24 and HLA-DR antigens (for the identification of macrophages) in biopsied cervix tissue of a woman having an HIV antigen-positive CVL sample. Panel A shows HIV p24 antigen-positive spots (arrows) and HLA-DR antigen-positive spots (other dark spots) were localized in the stroma. Panel B shows a control section stained without primary antibodies. Both tissue sections were counterstained with hematoxylin, magnification ×100.

FIG. 10 represents the micrographs of an HIV p24 antigen-positive biopsied cervix tissue of a woman with HIV antigen-positive CVL samples. Panel A shows HIV antigens localized in the epithelial layers (arrows). Panel B is a control section from the same tissue processed without the primary monoclonal HIV p24 antibody. Both tissue sections were counterstained with Fast Red, magnification ×200.

FIG. 11 represents the micrographs of an HIV p24 antigen-positive biopsied cervix tissue of a second woman with HIV antigen-positive CVL samples. Panel A shows HIV antigens localized in the epithelial layers (arrows). Panel B is a control section from the same tissue processed without the primary monoclonal HIV p24. Both tissue sections were counterstained with Fast Red, magnification ×200.

FIG. 12 represents the micrographs of cervix tissue of a woman with an HIV antigen-negative CVL sample that was processed with HIV p24 monoclonal antibody (Panel A) or in the absence of the HIV p24 monoclonal antibody (Panel B). Tissue sections were counterstained with Fast Red, magnification ×200.

### DETAILED DESCRIPTION OF THE INVENTION

This invention is based in part on the discovery that HIV can be detected in cervicovaginal lavage (CVL) from patients who are persistently seronegative (See Examples). Without being bound to any theory of the source or the implications of the HIV in CVL, it is believed that HIV is present in genital tissue before the blood when transmission occurs during sex. Screening of CVL for HIV could thus detect nascent HIV exposure and early HIV infections in women who are seronegative.

The invention is directed to methods of diagnosing HIV in a woman. The methods comprise obtaining a sample of genital tract cells and/or genital tract secretions from the woman; evaluating the sample for the presence of HIV; and determining whether the woman is HIV positive or HIV negative from the sample. The presence of HIV in the sample indicates the woman is HIV positive, and the absence of HIV in the sample indicates the woman is HIV negative.

These methods are not limited to the use of any particular method of obtaining a the sample of genital tract cells and/or genital tract secretions and includes at-home collection by the patient, as described in Nobbenhuis et al., 2002. Also, any technique of obtaining a Pap smear would be expected to be suitable for these methods. Specific collection methods contemplated include (but are not limited to) the spatula, cervicovaginal lavage (CVL), cytobrush and “Thin Prep” collection. Biopsied cervix tissue and endocervical curettage tissue are also contemplated. In some preferred embodiments, the sample is the result of a lavage using 10 ml saline. Preferably, the particulate matter is centrifuged from the sample to obtain a cell-free fraction and the assay is performed using the soluble supernatant fractions of the lavage samples. However, HIV viral proteins are also in the pelleted cells of the CVL samples (data not shown from Example 1).

In some aspects of these methods, the presence of HIV is determined by determining whether an HIV protein is present in the sample. Preferred HIV proteins for analysis here are p24 or gp41, but any HIV protein can be analyzed. See Example 1, mass spectrometry result section, in which additional HIV proteins that were detected in the CVL samples were described. These methods are not limited to detection of any particular HIV protein, including any form of Gag, Pol, Env, Nef, Vif, Rev, Vpr, Tat or Vpu.

The HIV protein can be detected by any method now known or later discovered, including methods using an antibody specific for the HIV protein. The antibodies can be monoclonal, polyclonal, or genetically engineered.

The antibody used here can also be one directed against epitopes on HIV proteins that are different in CVL than in serum. Methods for preparing such antibodies are known in the art. Since such antibodies would usually be raised against HIV antigen(s) specifically present in cervicovaginal milieu in the early phase of HIV infection, it is expected that the antibody would have higher affinity for the cervicovaginal-specific HIV protein(s) than the HIV protein that would appear in the blood at a later phase of HIV infection.

These antibodies can be used, for example, in any of the various immunoassays that do not require electrophoresis, for example ELISA, or immunoassays using dipsticks. Alternatively, the antibodies can be utilized in an immunoassay that employs electrophoresis, for example western blot assays.

The HIV protein can also be detected in the sample using methods that do not utilize an antibody, for example mass spectrometry, as in Example 1.

These methods can also be employed to detect a second (i.e., an additional) HIV protein. In these methods, the evaluation step (b) also comprises determining whether a second HIV protein is present in the sample. In some aspects, the second HIV protein is p24 or gp41. Alternatively, the second HIV protein can be any other HIV protein.

Additionally, or alternatively, the evaluation step (b) comprises determining whether an HIV protein and an HIV RNA is present in the sample.

Where HIV RNA is assayed, any known assay can be employed. Preferably, the HIV RNA is determined using reverse transcriptase-polymerase chain reaction (RT-PCR), a branched DNA assay, or nucleic acid sequence-based amplification (NASBA).

In these methods, the evaluation step (b) can comprise, or consist of, determining whether HIV RNA is present in the sample, for example using RT-PCR, a branched DNA assay, or NASBA.

These methods can also include testing peripheral blood serum from the woman for the presence of anti-HIV antibodies.

These methods can be utilized for the diagnosis of HIV in any woman, for example when a woman is brought into the health care system for a Pap smear and the Pap smear is diagnosed as Atypical Squamous Epithelial Cells of Unknown Significance (ASCUS). The methods can also be utilized for women who have cervical intraepithelial neoplasia (CIN), a term which depicts an abnormality in the epithelial layers of the cervix, also seen for in woman brought into the health care system on account of an abnormal Pap smear and on subsequent gynecologic examinations were found to have precursor cervix cancer lesions, termed as CIN. The CIN in these embodiments can be diagnosed as either Low Grade Squamous Intraepithelial Lesions (LGSIL) or High Grade Squamous Intraepithelial Lesions (HGSIL). These methods can also be used with women who have invasive cancer of the female genital tract.

In cases where the woman is diagnosed as having HIV due to the presence of HIV RNA and/or protein in the sample, the methods preferably further comprise recommending, and/or initiating anti-HIV therapy to the woman if HIV is present in the sample.

These methods can be used with any isolate or form of HIV, including any HIV-1, HIV-2, or any other HIV, now known or later discovered. Preferably, the HIV is HIV-1.

As established in the examples, an HIV seronegative woman can have HIV antigens and/or RNA in a genital tract sample. Thus, in some embodiments of these methods, the woman is HIV-seronegative.

These methods can further comprise recommending, and/or commencing, anti-HIV therapy to the woman if the HIV protein or RNA is present in the sample.

These methods could be used with any HIV now known or later discovered. Preferably, the HIV is HIV-1.

The invention is also directed to methods of diagnosing HIV in an HIV-seronegative woman. The methods comprise obtaining a sample of genital tract cells and/or genital tract secretions from the woman; evaluating the sample for the presence of HIV; and determining whether the woman is HIV positive or HIV negative from the sample. The presence of HIV in the sample indicates the woman is HIV positive, and the absence of HIV in the sample indicates the woman is HIV negative.

As in the methods described above, these methods are not limited to the use of any particular method of obtaining a the sample of genital tract cells and/or genital tract secretions and include any Pap smear, cervicovaginal lavage (CVL), cytobrush, Thin Prep collection, biopsied cervix tissue, and endocervical curettage tissue.

In some aspects of these methods, the presence of HIV is determined by determining whether an HIV protein is present in the sample, e.g., p24 or gp41, but any HIV protein can be analyzed.

The protein can be detected by any method now known or later discovered, including methods using an antibody specific for the HIV protein. The antibodies can be monoclonal, polyclonal, or genetically engineered.

The antibody used here can also be one directed against epitopes on HIV proteins that are different in genital tract cells or secretions than in serum.

These antibodies can be used, for example, in any of the various immunoassays that do not require electrophoresis, for example ELISA, or immunoassays using dipsticks. Alternatively, the antibodies can be utilized in an immunoassay that employs electrophoresis, for example western blot assays.

The HIV protein can also be detected in the sample using methods that do not utilize an antibody, for example mass spectrometry.

These methods can also be employed to detect a second (i.e., an additional) HIV protein. In these methods, the evaluation step (b) also comprises determining whether a second HIV protein is present in the sample. In some aspects, the second HIV protein is p24 or gp41. Alternatively, the second HIV protein can be any other HIV protein.

Additionally, or alternatively, the evaluation step (b) comprises determining whether an HIV protein and an HIV RNA is present in the sample.

Where HIV RNA is assayed, any known assay can be employed. Preferably, the HIV RNA is determined using reverse transcriptase-polymerase chain reaction (RT-PCR), a branched DNA assay, or nucleic acid sequence-based amplification (NASBA).

In these methods, the evaluation step (b) can comprise, or consist of, determining whether HIV RNA is present in the sample, for example using RT-PCR, a branched DNA assay, or NASBA.

These methods can be utilized on any woman in need of diagnosis for HIV, for example a woman who is diagnosed as having ASCUS or CIN, including LGSIL or HGSIL. These methods can also be used with women who have invasive cancer of the female genital tract.

In cases where the woman is diagnosed as having HIV due to the presence of HIV RNA and/or protein in the genital tract sample, the methods preferably further comprise recommending anti-HIV therapy to the woman if the HIV protein or RNA is present in the sample.

These methods can be used with any isolate or form of HIV, including any HIV-1, HIV-2, or any other HIV, now known or later discovered. Preferably, the HIV is HIV-1.

These methods can further comprise recommending, and/or initiating, anti-HIV therapy to the woman if HIV is present in the sample.

Additionally, the invention is directed to methods of determining whether to recommend that a woman should undergo anti-HIV therapy. The methods comprise obtaining a sample of genital tract cells and/or genital tract secretions from the woman; and evaluating the sample for the presence of HIV. The presence of HIV in the sample indicates that anti-HIV therapy should be recommended to the woman.

As in the methods described above, these methods are not limited to the use of any particular method of obtaining a the sample of genital tract cells and/or genital tract secretions and include any Pap smear, cervicovaginal lavage (CVL), cytobrush, Thin Prep collection, biopsied cervix tissue, and endocervical curettage tissue.

In some aspects of these methods, the presence of HIV is determined by determining whether an HIV protein is present in the sample, e.g., p24 or gp41, but any HIV protein can be analyzed.

The protein can be detected by any method now known or later discovered, including methods using an antibody specific for the HIV protein. The antibodies can be monoclonal, polyclonal, or genetically engineered.

The antibody used here can also be one directed against epitopes on HIV proteins that are different in CVL than in serum.

These antibodies can be used, for example, in any of the various immunoassays that do not require electrophoresis, for example ELISA, or immunoassays using dipsticks. Alternatively, the antibodies can be utilized in an immunoassay that employs electrophoresis, for example western blot assays.

The HIV protein can also be detected in the sample using methods that do not utilize an antibody, for example mass spectrometry.

These methods can also be employed to detect a second (additional) HIV protein. In these methods, the evaluation step (b) also comprises determining whether a second HIV protein is present in the sample. In some aspects, the second HIV protein is p24 or gp41. Alternatively, the second HIV protein can be any other HIV protein.

Additionally, or alternatively, the evaluation step (b) comprises determining whether an HIV protein and an HIV RNA is present in the sample.

Where HIV RNA is assayed, any known assay can be employed. Preferably, the HIV RNA is determined using reverse transcriptase-polymerase chain reaction (RT-PCR), a branched DNA assay, or nucleic acid sequence-based amplification (NASBA).

In these methods, the evaluation step (b) can comprise, or consist of, determining whether HIV RNA is present in the sample, for example using RT-PCR, a branched DNA assay, or NASBA.

These methods can also include testing peripheral blood serum from the woman for the presence of HIV-reactive antibodies.

These methods can be utilized on any woman in need of diagnosis for HIV, for example a woman who is diagnosed as having ASCUS or CIN, including LGSIL or HGSIL. These methods can also be used with women who have invasive cancer of the female genital tract.

These methods can be used with any isolate or form of HIV, including any HIV-1, HIV-2, or any other HIV, now known or later discovered. Preferably, the HIV is HIV-1.

These methods can further comprise initiating, anti-HIV therapy to the woman if HIV is present in the sample.

The woman tested can be either HIV-seropositive or HIV-seronegative.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims which follow the examples.

### EXAMPLE 1

### Identification of Human Immunodeficiency Virus (HIV) proteins in cervicovaginal lavage (CVL) samples from persistently HIV-seronegative women

Example Summary

Cervicovaginal lavage (CVL) allows sampling of cervical and vaginal cells and mucus. It is a routine procedure that is safe and minimally invasive, used mostly in gynecological clinics in the US monitoring for human papillomavirus (HPV) infection. In this study, investigated were cervix-specific proteins of the genital tract secretions of women with precursor lesions of the cervix, to determine whether the proteins correlated with CIN pathology. CVL sampling was used to retrieve the cervix-specific antigens from the genital tract. The twenty women recruited for the study had voluntarily entered the health care system to obtain Pap smears, which were diagnosed as abnormal. Colposcopically directed cervical biopsies were obtained subsequently to clarify the source of the abnormal cells, which were histopathologically diagnosed as cervical intraepithelial neoplasia. During the gynecological visit, the patients were recruited with informed contents. The study was approved by the Institutional Review Board (IRB). The patients were offered HIV testing as part of the patient care protocol, and all twenty women opted for it.

The cervix-specific antigens were to be monitored at three months interval for a period of one year. Matrix-assisted laser desorption ionization time of flight peptide mass finger printing (MALDI TOF PMF) mass spectroscopy was used to identify the proteins. HIV viral proteins p24 and gp41 were identified in the CVL samples of four women, of the twenty women studied. After the initial identification of HIV antigens in the CVL samples of these four women, CVL samples obtained from all twenty patients at follow-up clinic visits were also analyzed for HIV p24 and gp41 by western blot (WB). The monoclonal antibodies that were used were raised specifically against HIV-1 p24 and gp41 antigens, isolated from human serum of patients with AIDS. The CVL samples of the four women identified by mass spectrometry to be HIV antigen-positive were found to be positive for both HIV-1 p24 and gp41 by WB at each of their clinic visits; while for the remaining sixteen women, HIV viral antigens were not detected in their CVL samples by mass spectrometry or WB, at each clinic visit. During each clinic visit, a blood sample was also obtained from each patient, that were submitted to the hospital's HIV laboratory for HIV-serostatus assessments. The results as retrieved from the patients' clinical charts for all twenty patients, at each clinic visit, were reported to be negative for HIV-1 and HIV-2 antibodies.

To further clarify the presence of HIV antigens in the CVL samples, the samples were additionally analyzed for HIV p24 antigen by immunoaffinity chromatography. The chromatography results also documented HIV-1 p24 antigen to be present in the CVL samples of these four patients. The four women with HIV antigen-positive CVL samples are currently being followed. However, since their Pap smears were diagnosed as ASCUS (Atypical squamous epithelial cells of unknown significance) and they continue to remain seronegative, the patients are now being followed at intervals of one year instead of three months, according to American College of Obstetricians and Gynecologist's specifications.

Also explored was whether the HIV viral antigens were contributed by their male partners' semen. The semen contamination of the CVL samples was analyzed by examining the presence of prostate specific antigen (PSA) by WB. In the WB procedure, mouse monoclonal antibodies raised specifically against human prostate specific antigen, p30 was employed. The WB method used for the determination of PSA is considered to be more sensitive than detecting the PSA enzymatically, the procedure which is more often used in the determination of semen contamination of the CVL samples. The absence of PSA in HIV antigen-positive CVL samples confirmed that the HIV viral antigens in the CVL samples were not recent contributions of their male partners' semen.

Based on these findings it is concluded that some HIV-seronegative women can express HIV-1-specific proteins in the genital tract cells/secretions. Such women may have true mucosal HIV infection without systemic evidence, aborted HIV infection with presence of some residual protein, or early HIV infection unaccompanied by a systemic antibody response. The findings more importantly demonstrate that CVL sampling could provide a unique opportunity for monitoring HIV exposure and early HIV infection in asymptomatic women, who are unaware of their HIV status, prior to seroconversion.

Introduction

The present proteomics study was undertaken to identify HPV viral proteins in the CVL samples that would correlate with CIN pathology based on the role HPV plays in CIN and cervical cancer.

Methods

Subject recruitment. Twenty asymptomatic women with abnormal Pap smears and biopsy-proven CIN grade 1 lesions were recruited with informed consent from a public hospital in the Bronx, New York. Each woman completed a brief study questionnaire including age, last menstrual period, smoking history, contraceptive practices and the date and results of her last Pap smear. The study protocol was approved by the Institutional Review Board (IRB) of the Albert Einstein College of Medicine.

Collection and processing of CVL samples. After we obtained informed consent we collected a CVL specimen from each volunteer participant. Half of the lavage sample was collected in a tube containing a cocktail of protease inhibitors (Roche, Indianapolis, Ind.) for the Proteomic study. The other half of the CVL was used for the determination of human papillomavirus (HPV) DNA by polymerase chain reaction (PCR) methods, using consensus primer MY09 and MY11 to a highly conserved region in the L1 open reading frame (Castle et al., 2002).

The CVL samples were placed in ice and brought back to the laboratory within two hours of collection. For protein analyses, CVL specimens were processed in the laboratory the same day of sample collection. Samples were centrifuged at 229,000 xg in a Beckman Ultracentrifuge (TLA 120.2) at 4 0 C for 20 minutes, to remove all particulate matters. The soluble supernatant fractions were stored at −20 0C in aliquots and were used in the study.

The inventors have employed CVL sampling in many epidemiological studies conducted to delineate the risk factors associated with the pathogenesis of CIN and cervical cancer in the absence or presence of Human Papillomavirus (HPV) infections (Basu et al., 1993; 2005).

Polyacrylamide gel electrophoresis and mass spectrometry of CVL samples. Protein concentrations of the soluble supernatant fractions of the CVL samples were determined using Nano prop ND-1000 spectrophotometer (NanoDrop Technologies, Inc., Wilmington, Del.). 20 μl aliquots of the CVL samples were loaded onto precast 10% Tris-HCl polyacrylamide gels (BioRad, Hercules, Calif.) along with protein markers (Rainbow marker 800, GE Healthcare, Piscataway, N.J.). Following electrophoresis, the gels were either stained with Coomassie Brilliant blue or electroblotted onto nitrocellulose membranes (Schleicher Schuell Bioscience, Inc., Keene, N.H.) for Western Blot analysis.

Multiple Coomassie stained polypeptide bands were seen in each of the lanes of the gels. The stained polypeptide bands were excised when the gels were still wet and were processed for in-gel trypsin digestion. Trypsin-digested peptides were analyzed by MALDI TOF PMF. Portions of the gel from adjacent lane(s) that were free of CVL samples but corresponded to the regions of the Coomassie stained polypeptide bands were cut and processed simultaneously to serve as controls. Prior to mass spectrometry, the tryptic peptides were purified using either the C¹⁸ Ziptips (Millipore Corporation, Bedford, Mass.) or PepClean C-18 Spin Columns (Pierce, Rockford, Ill.), as per the manufacturers' instructions. Mass spectrometry was performed using equal volumes (1.5 μl each) of the purified peptide mixture and matrix solution (saturated α-cyano-4 hydroxycinnamic acid in 50% acetonitrile containing 0.1% TFA), and 1.5 μl of the resultant mixture was spotted onto matrix-assisted laser desorption ionization (MALDI) target plates. The samples were analyzed using a Voyager-DE STR MALDI TOF mass spectrometer (Applied Biosystems, Foster City, Calif.). Mass spectra were recorded in positive ion reflector mode at an average resolution of 4500. One hundred shots were summed to obtain the spectrum. Protonated molecular ions (M+H)⁺ of three trypsin autodigestion peptides (m/z 842.51, 1045.56, 2211.10), and a matrix peak (m/z 568.14) were used as internal calibrants. Unique monoisotopic peaks (m/z values) with distinct peptide isotopic envelopes, not seen in the control spectra, were selected for PMF. For protein identification, ProFound™ and MASCOT™ (in-house) were used to search the database. The taxonomic categories used for protein identification were: “All Taxa”, “Homo sapiens” and “Virus”. Protein identification in the present study was based on the guidelines in publications of peptide and data (Carr 2004).

Western blot assays. We resolved 20 μl aliquots of the CVL samples on 10% Tris-HCl gels for 1 D SDS PAGE. Proteins were transferred onto nitrocellulose membranes. Unspecific protein binding was blocked with 3% milk in Tris-buffered saline (pH 7.4), containing 0.1% Tween-20 (TBST). The blots were then individually probed with specific mouse MABs for one hour at room temperature. After thoroughly washing the blots with TBST four times for 15 minutes each at room temperature, the blots were probed with a secondary antibody for an hour. The blots were again thoroughly washed with TBST four times for 15 minutes each at room temperature, and the immunoreactive protein bands were then visualized using enhanced chemiluminescent reagent (GE Healthcare, Piscataway, N.J.) as per manufacturer's direction. The dilution of the primary antibodies varied between 1:500 and 1:800. The secondary antibody was used at a dilution of 1:3000. The following antibodies were used for the various WB experiments (i) HIV-1 p24 MAB (from three separate commercial vendors: Abcam Inc. Cambridge, Mass.; Cliniqa, Fallbrook, Calif.; ZeptoMetrix, Buffalo, N.Y.; and one that was obtained courtesy of Dr. Shibo Jian, of New York Blood Bank, NY); (ii) recombinant full length HIV-1 p24 MAB (BioWorld, Dublin, Ohio); (iii) env glycoprotein gp41MAB (ZeptoMetrix, Buffalo, N.Y.), and (iv) phosphotyrosine MAB, recombinant 4G10 (Upstate Cell Signaling Inc., San Francisco, Calif.). The secondary antibody was a goat anti-mouse horseradish peroxidase conjugated polyclonal antibody (BD Biosciences, Franklin Lakes, N.J.).

The western blot analyses were performed as follows:

(a): To confirm the presence of HIV-1 p24 and gp41 antigens in the CVL samples. The mouse MABs used were raised against specific human HIV-1 p24 and HIV-1 gp41 antigens isolated from serum samples of patients with AIDS. 20 μl aliquots of CVL samples in duplicates were resolved on a gel. Following SDS PAGE, each gel was divided into two, half of the gel was stained with Coomassie blue to examine the protein profile. The proteins of the other half of the gel were transferred onto nitrocellulose membrane and the immunoblots were then probed with either HIV-1 p24 MAB (1:500) or gp41 MAB (1:500) to detect the presence of the HIV antigens.

(b) Since immunoglobulins are inherent constituents of CVL samples and on SDS PAGE can yield 25 kDa and 50 kDa fractions, WB assay was carried out to examine whether the immunoreactive bands noted were due to immunoglobulins. 20 μl aliquots of four CVL samples were resolved on a gel, in duplicates. One half of the immunoblot was probed with HIV-1 p24 MAB followed with a secondary antibody. The remaining half of the immunoblot was probed with the secondary antibody alone.

(c) An immunoaffinity chromatographic procedure was carried out using the Microlink Protein Coupling Kit (Pierce, Rockford, Ill.) to reconfirm the presence of HIV-1 p24 in the CVL sample. First a phosphotyrosine MAB conjugated immunoaffinity column was prepared. 160 μl of a CVL sample was loaded on to the column. After washing the column to remove the unbound proteins and other constituents present in the CVL sample the antibody-bound protein was then eluted from the column, and WB assay was performed to determine the presence of HIV-1 p24 antigen in the eluate.

(d) Since the immunoreactive bands seen in WB assays for HIV-1 p24 MABs positive CVL samples repeatedly corresponded to 25 kDa molecular weight, while the immunoreactive positive band depicting the full length recombinant HIV-1 p24 protein (BioWorld) corresponded to 24 kDa, we examined whether the HIV-1 p24 protein in the CVL was phosphorylated. An immunoblot that had identified the presence of HIV-1 p24 antigen in the CVL samples was first stripped to remove the HIV-1 p24 MAB from the blot, and then the immunoblot was reprobed with Phosphotyrosine MAB, recombinant 4G10 (Upstate Cell Signaling Inc., San Francisco, Calif.) to determine the presence of phosphotyrosine in the analyzed CVL samples.

(e) To determine whether the HIV viral proteins were from their male partners' semen we examined the CVL samples for the presence of prostate specific antigen (PSA). The mouse MAB used for these WB assays was raised against human PSA corresponding to amino acid 1-261 of a full length PSA p30 (Santa Cruz Biotechnology, Inc., Santa Cruz, Calif.). Aliquots of HIV-1 p24 positive and negative CVL samples were resolved on a gel. The proteins were transferred to immunoblots and probed with PSA MAB to determine the presence of PSA.

(f) Two immunoreactive bands were consistently noted in each HIV-1 p24 positive CVL sample: one at 25 kDa, and the other at the 41 kDa regions, although one single immunoreactive band was expected that would correspond to the 24 kDa molecular weight. To clarify whether the p41 band was an artifact, the immunoblots were probed with four HIV-1 p24 MABs, obtained from four different vendors and a fourth HIV-1 p24 MAB that was obtained from the New York Blood Center. For the experiment, four 20 μl aliquots of the same CVL sample were resolved on a gel. The proteins were transferred onto a nitrocellulose membrane. The immunoblot was then split into four sections, and each section was probed with a p24 MAB, obtained from a different source (Abcam Inc. Cambridge, Mass.; or Cliniqa, Fallbrook, Calif.; or ZeptoMetrix, Buffalo, N.Y.; or Dr. Shibo Jian, NY Blood Bank, NY).

Results

All twenty women in the study had histopathologically diagnosed CIN 1 lesions. On the day the CVL samples were collected, the Pap smear results ranged from “within normal limits” through high grade squamous intraepithelial lesions (HGSIL). HPV DNA was detected in 11 out of 20 (55%) women and were categorized as follows: high risk types 16, 18, 33, 35, 39, and 53; low risk type 72, and unknown risk type 62 (Table 1). The age of the women ranged from 18 to 50 years. Four women in the study were identified to have HIV antigens. The mean age of women with genital tract HIV proteins was found to be 43.2 years ±5.1 SD (n=4) compared to 26.2 years ±6.9 SD for women without genital tract HIV proteins (n=16; p=0.002 by Student's t test, assuming unequal variances). Only two women in the study were smokers. Eight women in the study were on oral contraceptive pills, eight used condoms, three have had tubal ligations, and one had an intrauterine device (IUD). Based on their hospital medical charts, all 20 women were negative for both HIV-1 and HIV-2 antibodies. The four women in the study are still asympotomatic and continue to remain seronegative for HIV.

TABLE 1
Demographic characteristics of patients

| | | | | | | | | HIV-1 p24 in |
|---|---|---|---|---|---|---|---|---|
| | | | LMP | ²Smoking | ³Contraception | HPV DNA | ⁴PSA status | CVL (MALDI |
| Patient | Age | ¹Pap Result | days | Status | Status | in CVL | of CVL | TOF PMF) |
| 1 | 46 | LGSIL | 23 | Non | Tubal | Positive | Positive | Positive |
| | | | | Smoker | Ligation | Type 58 | | |
| 2 | 27 | ASCUS | 21 | Non | Condom | Positive | Negative | Negative |
| | | | | Smoker | | Type 16 | | |
| 3 | 37 | ASCUS | 9 | Non | Tubal | Negative | Positive | Positive |
| | | | | Smoker | Ligation | | | |
| 4 | 22 | Negative | 30 | Non | Intrauterine | Negative | Not | Negative |
| | | for CIN | | Smoker | Device | | Analyzed | |
| 5 | 25 | LGSIL | 8 | Non | Condom | Positive | Negative | Negative |
| | | | | Smoker | | Type 72 | | |
| 6 | 50 | LGSIL | 40 | Non | Condom | Positive | Negative | Positive |
| | | | | Smoker | | Type 39 | | |
| 7 | 34 | LGSIL | 7 | Non | Condom | Negative | Positive | Negative |
| | | | | Smoker | | | | |
| 8 | 46 | HGSIL | 12 | Smoker | Condom | Negative | Negative | Negative |
| 9 | 20 | LGSIL | 4 | Non | Condom | Positive | Positive | Negative |
| | | | | Smoker | | Type 18 | | |
| 10 | 25 | ASCUS | 15 | Non | Pills | Positive | Positive | Negative |
| | | | | Smoker | | Type 33 | | |
| 11 | 22 | ASCUS | 77 | Non | Pills | Positive | Negative | Negative |
| | | | | Smoker | | Type 59 | | |
| 12 | 21 | ASCUS | 30 | Non | Pills | Positive | Negative | Negative |
| | | | | Smoker | | Type 16 | | |
| 13 | 21 | ASCUS | 62 | Non | Pills | Negative | Positive | Negative |
| | | | | Smoker | | | | |
| 14 | 23 | Negative | 8 | Smoker | Pills | Negative | Not | Negative |
| | | for CIN | | | | | Analyzed | |
| 15 | 40 | LGSIL | 9 | Non | Pills | Negative | Negative | Positive |
| | | | | Smoker | | | | |
| 16 | 25 | HGSIL | 90 | Non | Pills | Positive | Not | Negative |
| | | | | Smoker | | Type 62 | Analyzed | |
| 17 | 31 | ASCUS | 25 | Non | Condom | Negative | Negative | Negative |
| | | | | Smoker | | | | |
| 18 | 18 | ASCUS | 24 | Non | Condom | Positive | Not | Negative |
| | | | | Smoker | | Type 16 | Analyzed | |
| 19 | 36 | Negative | 6 | Non | Tubal | Negative | Positive | Negative |
| | | for CIN | | Smoker | Ligation | | | |
| 20 | 24 | Negative | 40 | Non | Pills | Positive | Negative | Negative |
| | | for CIN | | Smoker | | Type 16 | | |

LGSIL/HGSIL = Low/High grade squamous intraepithelial lesion; ASCUS = Atypical cells of unknown significance.
²Smokers smoked ½ to 2 packs a day for at least 1 year.
³Women on pills were at least for a period of six months and were on various forms.
⁴The PSA status of 4 CVL samples was not determined due to insufficient samples.

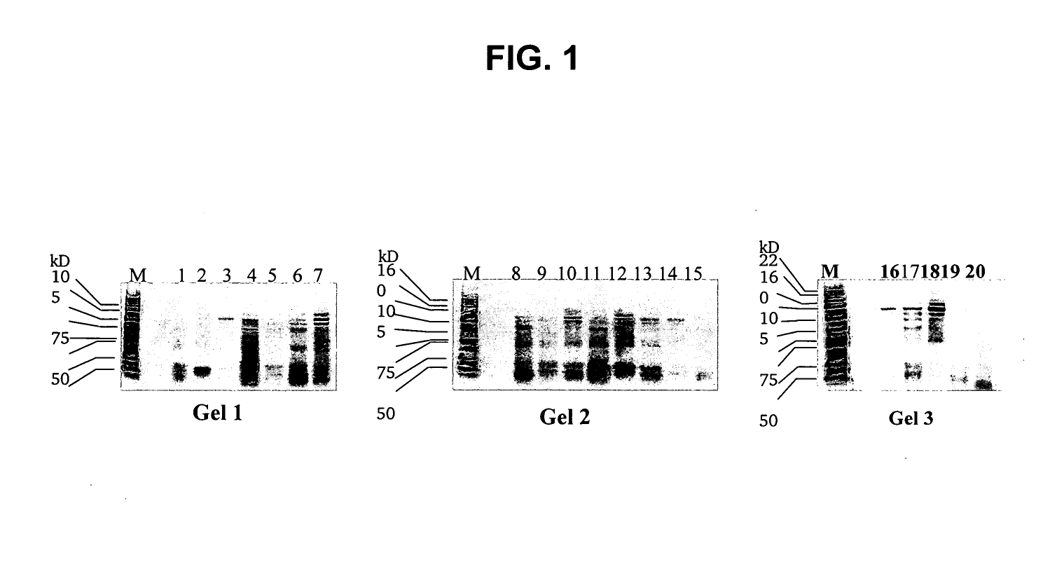

Protein expression profiling of CVL samples including mass spectrometry. The protein concentrations of the 20 samples ranged from 0.18-1.34 mg/ml. Due to these wide variations in protein concentrations among the CVL samples, the samples could not be normalized prior to electrophoresis, and 20 μl aliquots of the lavage samples were analyzed by ID SDS-PAGE. The gel patterns (FIG. 1) showed distinct polypeptide bands in the CVL samples across a wide range of molecular weights, with marked variations in the protein profiles among the CVL samples.

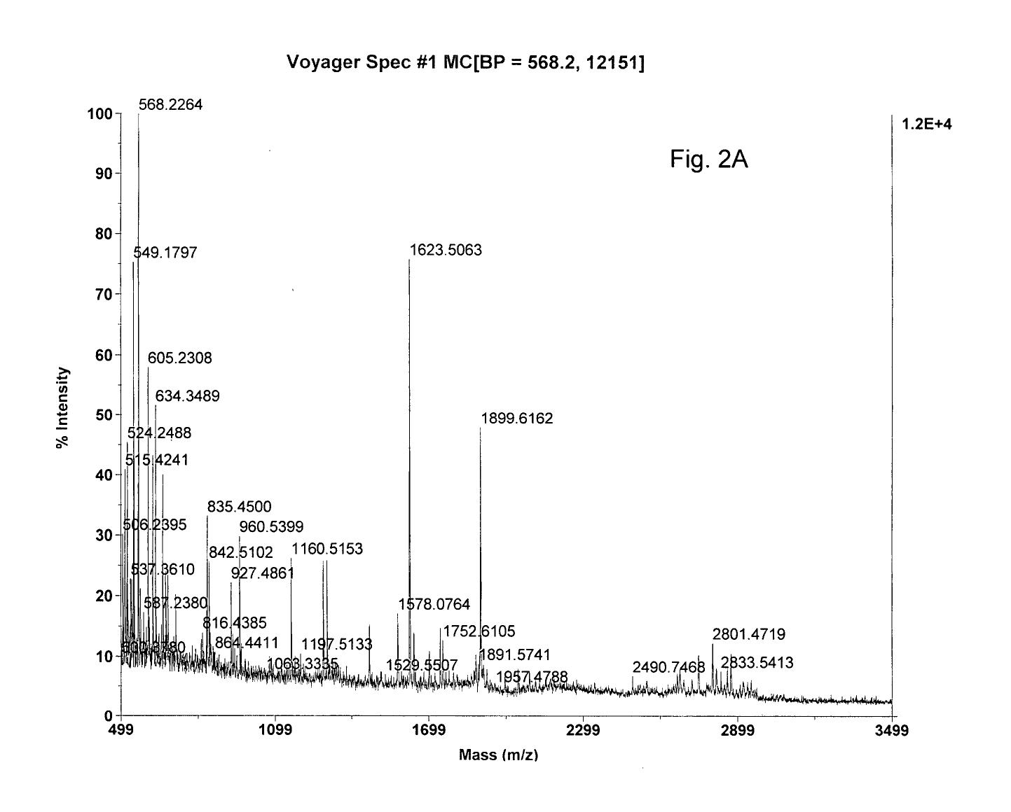

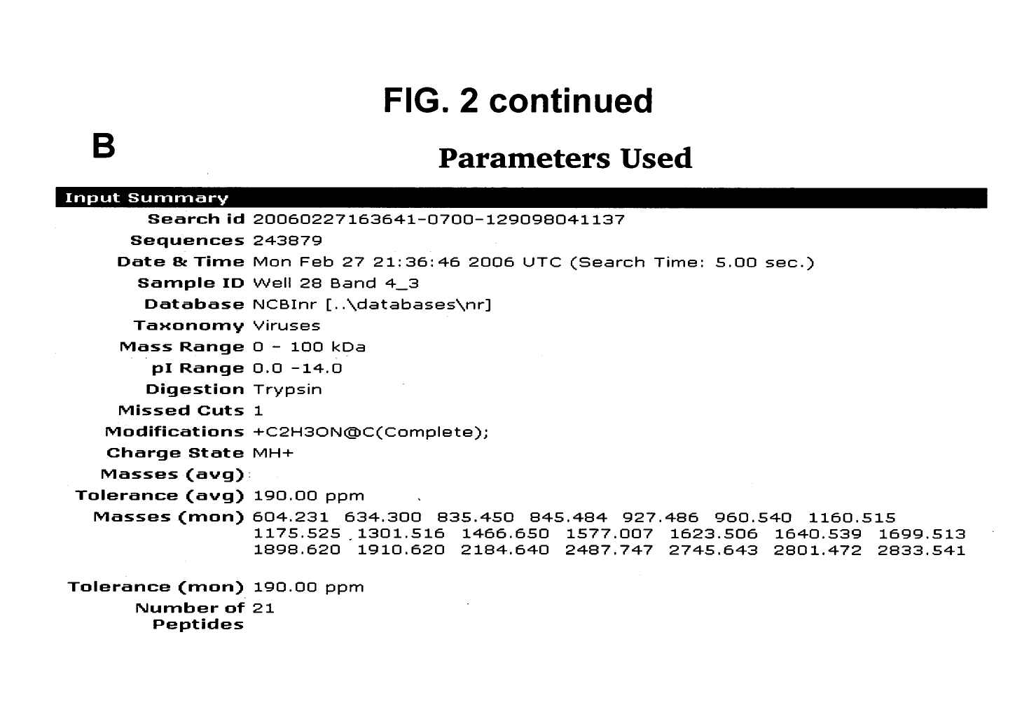

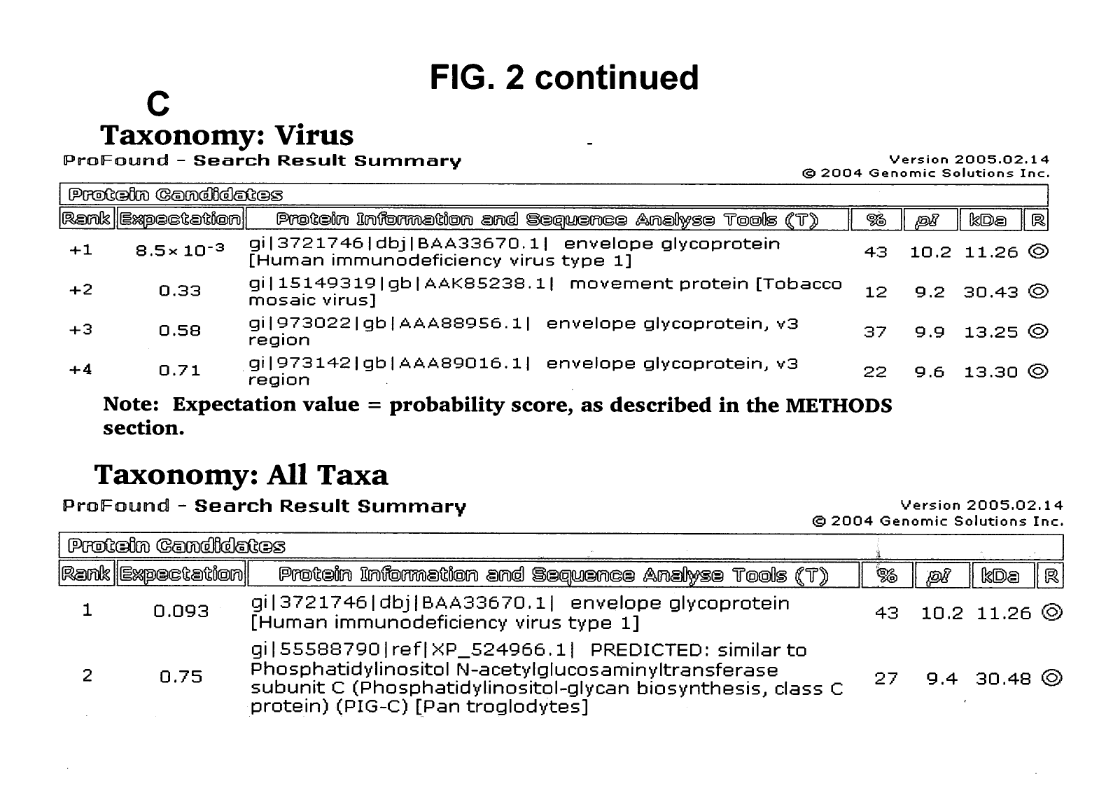

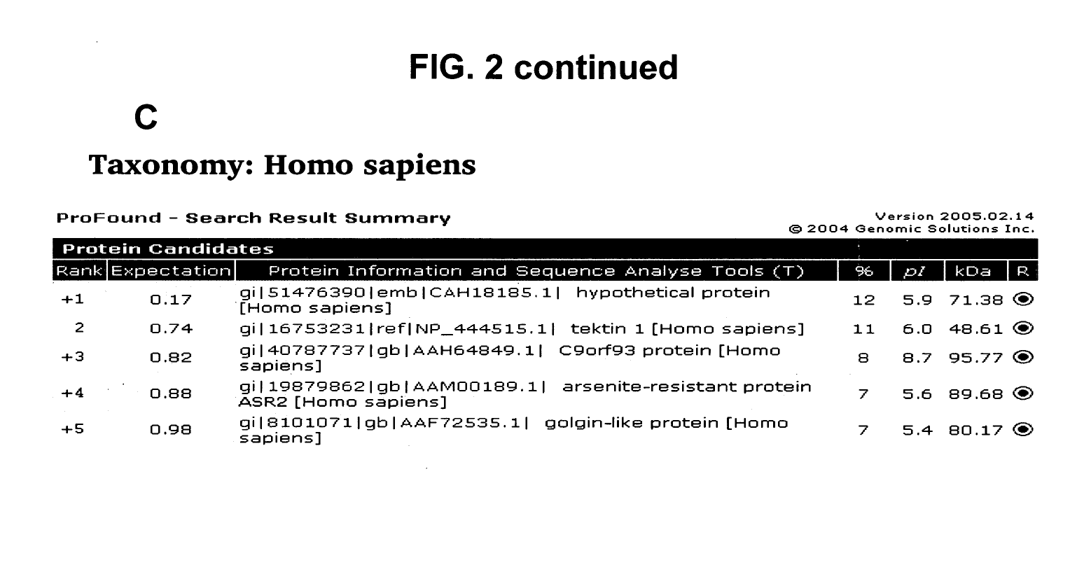

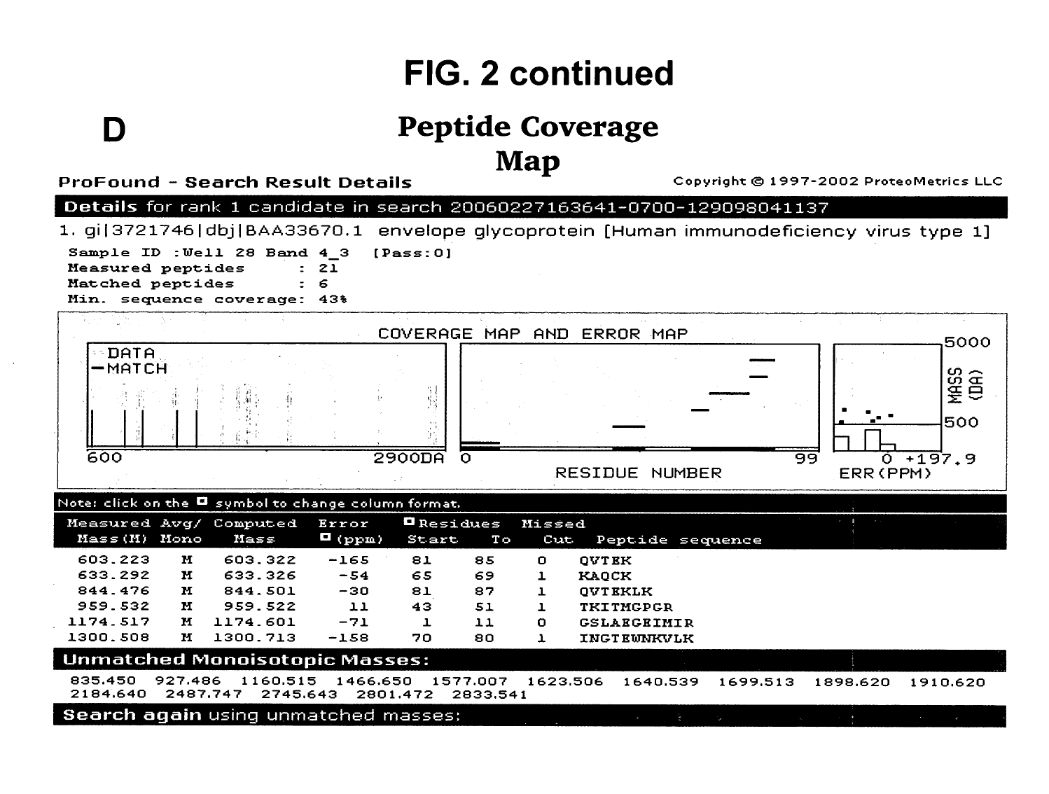

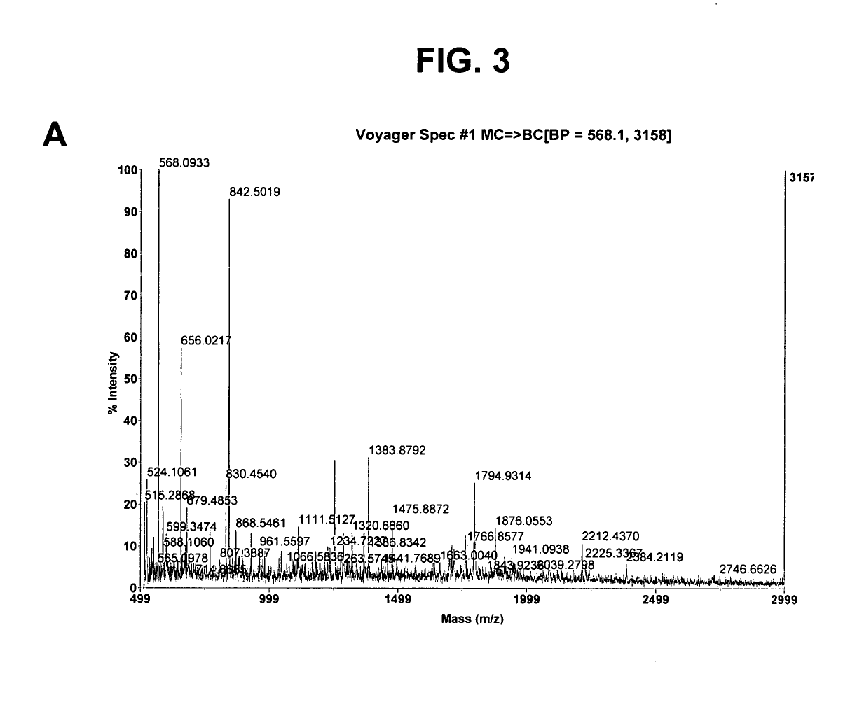

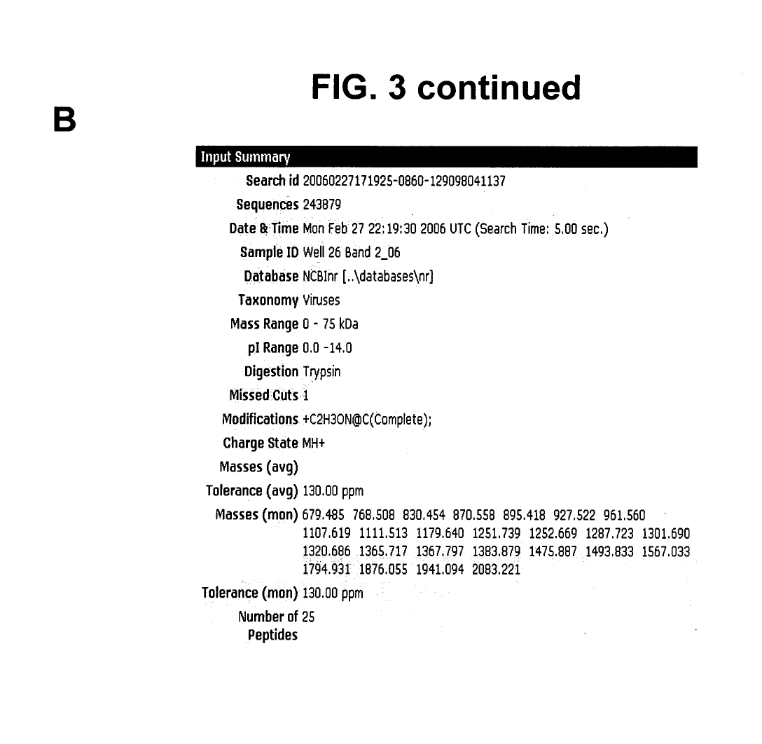

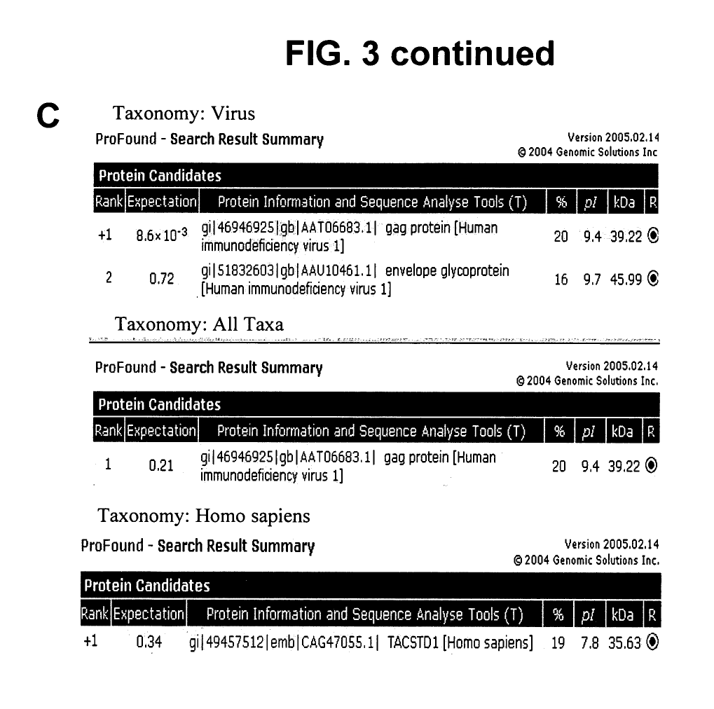

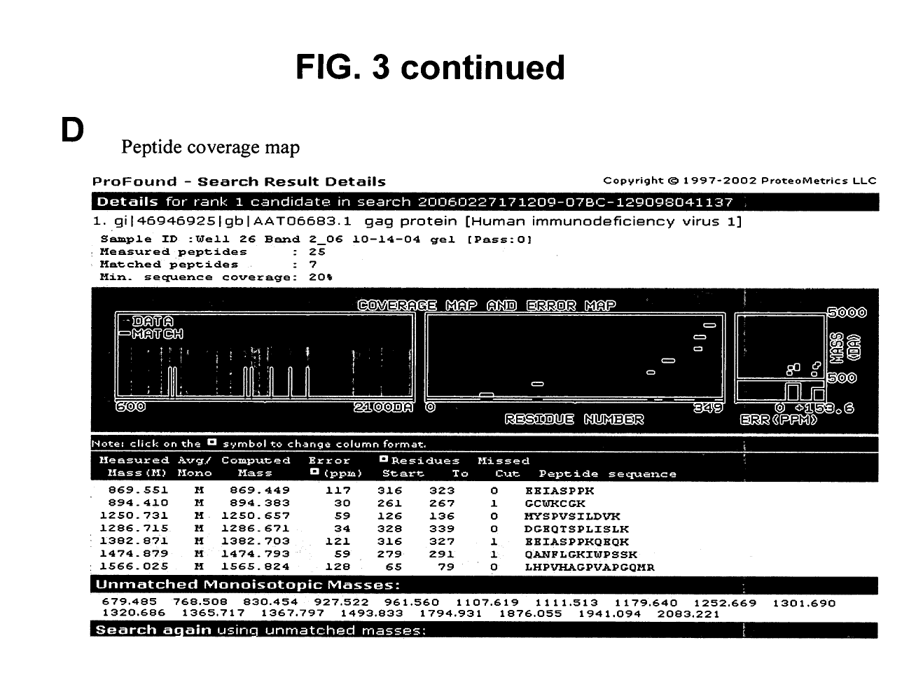

Numerous Coomassie-stained polypeptide bands were excised from the ID gels of 20 CVL samples. Each polypeptide band was digested with trypsin followed with MALDI TOF PMF for protein identification. Mass spectrometry identified HIV-1 gag and env glycoprotein in four out of 20 CVL samples. The other proteins that were identified in the various polypeptide bands of the CVL samples included HPV-related early and late proteins and Hepatitis C viral proteins; and in the Homo sapiens category included: albumin, IgG heavy and light chains, T cell receptor β-chain, MHC Class 11 antigen, Alpha2 macroglobulin precursor, Beta Globin chain, Keratin, and Sperm associated antigen. The MALDI TOF mass spectrometry data of two representative polypeptide bands: one in which HIV-1 env glycoprotein and the other in which HIV-1 gag protein were identified are shown in FIGS. 2 and 3, respectively.

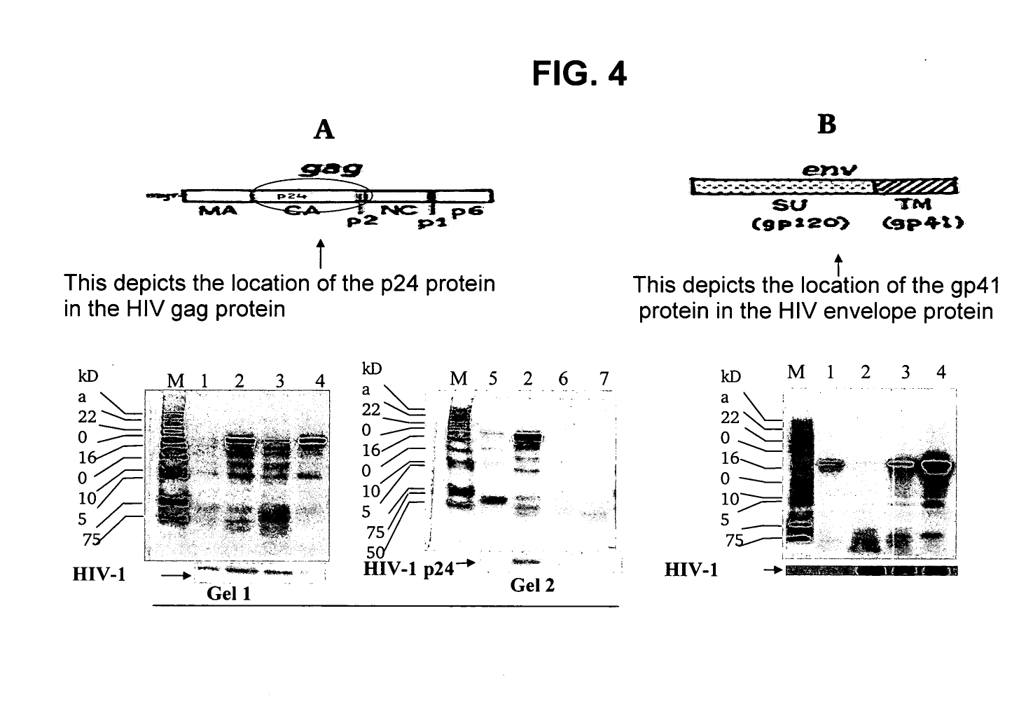

Western blot analysis to confirm the presence of HIV viral proteins. Coomassie-stained protein profiles and their corresponding WB data depicting HIV-1 p24 and gp41 positive and negative CVL samples are presented in FIGS. 4a and 4b, respectively. The WB data in Gel1 (FIG. 4A) represent four HIV p24 positive CVL samples, and Gel2 represent three HIV p24 negative and one HIV p24 positive CVL samples, respectively. Lanes 3 of both Gel1 and Gel2 in FIG. 4A represent the same CVL sample. The WB data shown in FIG. 4B demonstrate one negative and three HIV-1 gp41 positive CVL samples. Although Coomassie-stained protein profiles in lane 5 of Gel 2 (sample #7, FIG. 4A) and lane 2 of FIG. 4B show distinct polypeptides bands, however, their corresponding WB data demonstrate an absence of HIV-1 p24 and gp41 antigens. This suggests that p24 or gp41 positivity of the CVL samples is not dependent on the protein concentrations.

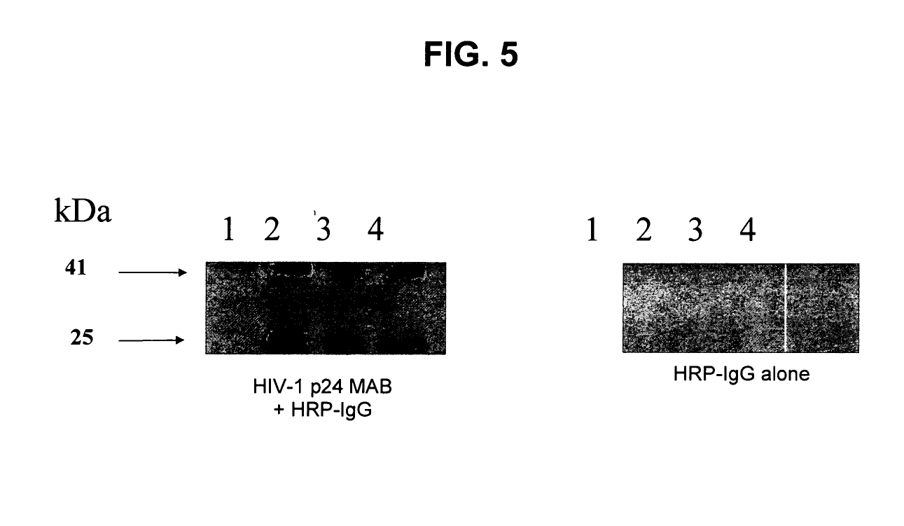

Examination of the contribution of inherent Immunoglobulin of CVL samples. The WB results shown in the left panel of FIG. 5 demonstrate that among the four CVL samples analyzed, one sample was negative (lane 1) and the remaining three were positive for HIV-1 p24 (lanes 2-4). The right panel of FIG. 5 depicts the immunoblot that was probed with the secondary antibody alone. The absence of immunoreactive bands when the immunoblot was probed with the secondary antibody alone indicated that the immunoglobulins in the CVL samples were not responsible.

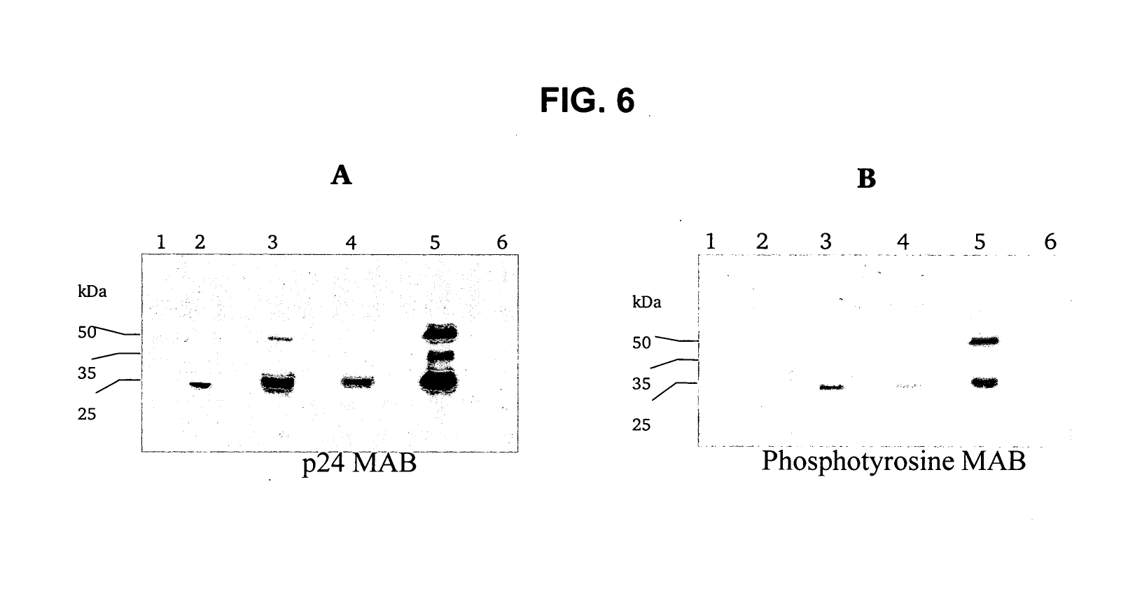

Reconfirmation of the presence of HIV-1 p24 by IC. FIG. 6A depicts the immunoblot that was probed with HIV-1 p24 MAB. The immunoreactive bands can be noted in lanes 2, 3, 4 and 5 that corresponded to 25 kDa. In the lane in which the control immunoaffinity column eluate was loaded, (lane 6), no immunoreactive band was noted. An additional immunoreactive band was noted in each of lanes 3, 4 and 5, at a region that corresponded to 41 kDa molecular weight. Similar bands at the 41 kDa region were also noted in all immunoblots probed with HIV-1 p24 MAB that may exhibit HIV-1 gag p41. In lane 5, an additional band was noted at a region corresponding to approximately 55 kDa, which probably could be a dimer of HIV-1 p24 and 41 gag proteins. This p55 protein noted in the lane experimental affinity column eluate was loaded (lane 5) could only be apparent when enough CVL sample protein was loaded onto the lane (160 μl) and was not apparent when only 20 μl of the soluble protein fraction of the CVL sample was loaded as in each of the lanes 3 and 4.

The difference in the electrophoretic migration of the HIV-1 p24 recombinant protein band (control) and the HIV-1 p24 band of the CVL samples noted in many of the WB results suggested that the HIV-1 p24 protein in the CVL sample could be phosphorylated. Hence, the HIV-1 p24 MAB was stripped from the above immunoblot and the blot, was then probed with phosphotyrosine MAB. The immunoreactive bands seen are presented in FIG. 6B. In lanes 3, 4 and 5, that corresponded to the three sample lanes (FIG. 6B) immunoreactive bands were visible. What was intriguing is that the immunopositive bands noted with phosphotyrosine MAB, were exactly at the regions where the HIV p24 positive bands were previously seen, (the bands exactly matched when the two autoradiographs were placed on top of each other), indicating that the p24 antigen in the CVL sample was indeed phosphorylated. In lane 2, the immunopositive band that corresponded to the recombinant HIV-1 p24, seen clearly in the previous autoradiograph, was no longer seen in the immunoblot probed with phosphotyrosine MAB. When the immunoblot was further stripped and probed with secondary antibody alone, no immunoreactive band was observed in any of the lanes. Collectively the experiments presented in FIG. 6 confirm that (a) the CVL sample was positive for HIV-1 p24, (b) the HIV-1 p24 antigen in the CVL sample was phosphorylated and, (c) the immunoglobulins in CVL samples were not responsible for the immunoreactive bands.

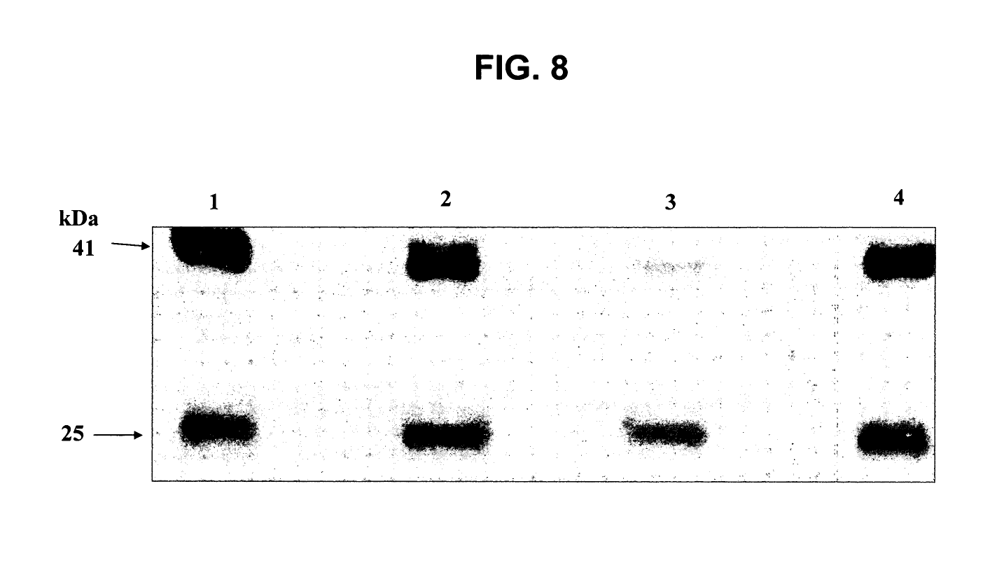

Clarifying the presence of 41 kDa protein in immunoblots probed with HIV-1 p24 MAB. All immunoblots of HIV-1 p24 positive CVL samples probed with HIV-1 p24 MAB demonstrated two immunoreactive bands (25 kDa and 41 kDa) instead of one at the 24 kDa region. That the p41 band seen was not an artifact could be evident from the WB data presented in FIG. 8. The figure demonstrates that irrespective of the source of HIV-1 p24 MAB used, two immunoreactive bands could be noted in each of the four nitrocellulose strips, one corresponding to 25 kDa and the other to 41 kDa molecular weights.

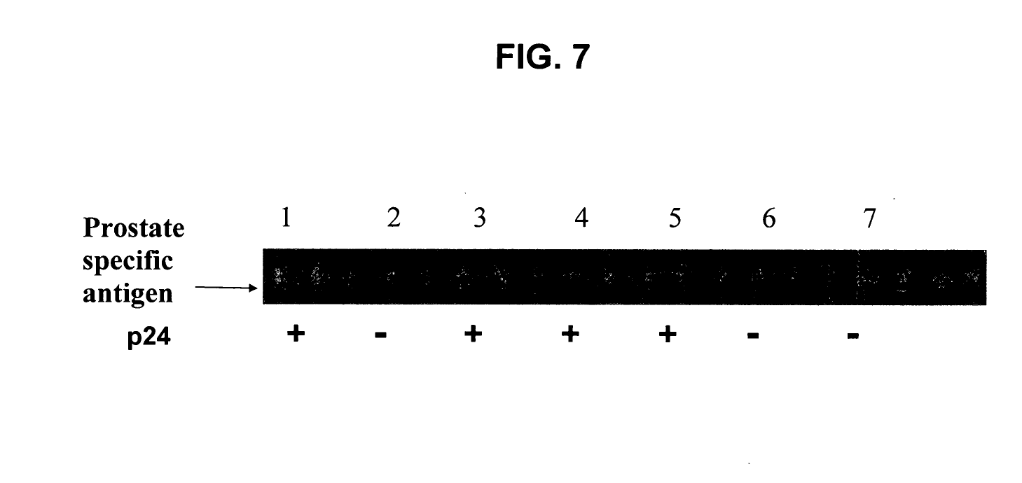

Semen contamination of CVL. The PSA status of all 20 CVL samples is presented in Table 1. Although PSA positive bands were noted in 7 out of 20 CVL samples analyzed (Table 1), the absence of PSA in lanes 6 and 7 that were positive for HIV-1 p24 (FIG. 7), suggest that HIV-1 p24 in the CVL samples was not contributed by semen contamination.

Discussion

In this study, among 20 women in the Bronx, New York, MALDI TOF PMF results identified HIV gag and env glycoprotein in the CVL samples of four women, who were seronegative for HIV. The presence of these HIV proteins in the four women was established in several different experiments. The identification of HIV-1 gag and HIV-1 env glycoprotein in four out of 20 women by MALDI TOF PMF was based on the protein identification criteria as specified by the guidelines published for mass spectrometry protein identification (Carr et al., 2004). The MALDI TOF PMF criteria that the guideline specified to be disclosed include: an annotated mass spectrum, peak lists, the number of matched peaks, the number of unmatched peaks, the sequence coverage, the score for highest rank hits, mass accuracy, resolution, the method how calibration was achieved, parameters used, threshold used to analyze the data and exclusion of known contaminating ions. Moreover, the presence of the viral proteins in the CVL samples were additionally confirmed by WB analysis, using mouse monoclonal antibodies raised against human HIV-1 p24 and gp41 antigens. Furthermore, the presence of HIV-1 p24 antigen in the CVL was established using an additional immunoaffinity procedure. The HIV p24 protein in the CVL samples was found to migrate differently than recombinant HIV p24. Our results show that the p24 protein in the CVL samples was phosphorylated. Posttranslational modifications of HIV p24 on serine and tyrosine residues have been reported by other investigators in separate in vitro studies (Laurent et al., 1989; Veronese et al., 1988). That 20% of the studied women with CIN should have HIV proteins in their CVL samples was especially unexpected, since the background HIV seroprevalence rates among women in the Bronx coming for out-patient care is likely <6%. The increased percentage of HIV infection detected in our study could be attributed to the difference in the criterion used in the detection of HIV; the use of antigen in the genital tract cells and secretions as opposed to the reliance of HIV antibodies to appear in the blood.

Since proteins are transient molecules that are easily degraded in the female genital tract, the present findings suggest the following possibilities for the 20% of our sample of women: (i) some HIV-seronegative women can express HIV-1-specific proteins in their genital tract cells and/or secretions via mucosal infection without systemic manifestations, (ii) some HIV-seronegative women were in the process of acute seroconversion, expressing protein in the mucosal tissues, (iii) some women had some form of aborted HIV infection with some residual protein presence, or (iv) some women had sex with an HIV-infected man from whom the protein was derived. While others have reported evidence of mucosal, but not systemic infection, and/or evidence of repeated HIV exposure without infection, such studies were conducted not with asymptomatic individuals unaware of their HIV status, but with patients in populations at high risk for HIV. (Becker, 2005; Berger et al., 1999; Bleul et al., 1997; Cocchi et al., 1995; Devito et al., 2000; Dorrell et al., 2001; Dragic et al., 1996; Kunanusont et al., 1995; Liu et al., 1996; McNeeley et al., 1997; Naif et al., 1998; Samson et al., 1996; Doumas et al., 2005; Stranford et al., 1999; Trabattoni et al., 2004; Yang et al., 1997; Zhou et al., 2002).

Many steps in HIV infection have been elucidated in recent years. To enter cells, HIV binds to CCR5 and CXCR4 co-receptors in CD4-bearing host cells, e.g., CD4 T lymphocytes, macrophage and dendritic cells (Berger et al., 1999; Dragic et al., 1996). The macrophage-tropic (M-tropic) HIV strains have a nonsyncytium-inducing phenotype and predominate during the early phase of HIV infection. Macrophage inflammatory protein 1α (MP1 α), MP1β and regulated on activation normal T expressed and secreted (RANTES) are host's natural proteins that have ligands for CCR5; by binding to CCR5, these chemokines block the M-tropic strains of HIV from entering CD4 T lymphocytes (Berger et al., 1999; Dragic et al., 1996). As the M-tropic strains of HIV in the hosts mutate and shift to syncytium inducing types, T lymphocyte-tropic HIV strains emerge that have increased cytopathogenicity and result in a more rapid depletion of T cells and progression of HIV disease (Berger et al., 1999; Dragic et al., 1996). The chemokine Stromal Cell Derived Factor 1 on the other hand, has ligands for CXCR4 and results in anti-HIV infectivity of the T-tropic HIV strains (Berger et al., 1999; Bleul et al., 1997; Dragic et al., 1996).

Host genetic variability is a major component determining the fate of individuals exposed to various pathogens. In HIV-exposed uninfected individuals, resistance to HIV infection has been attributed to inheritance of a genetic mutation in the CCR5 gene that has a 32-basepair deletion. The mutated CCR5 protein expressed by these genes lacks 32 amino acids, which disallow the CCR5 protein to be expressed on cell surfaces, rendering the CCR5 co-receptors nonfunctional, thereby preventing the HIV strains from entering into CD4 cells (Liu et al., 1996). However, it is reported that the CCR5 allele (^{32▴/32▴}) is almost always absent among people of African or Asian origins and among Caucasians; the frequency is ˜1% (Samson et al., 1996). This suggests that the mutation of CCR5 (^{32▴/32▴}) allele could not solely be responsible for conferring protection to HIV infection. The presence of anti-CCR5 antibodies in the mucosa of genital tract of HIV exposed but uninfected individuals is reported to elicit antigen down-modulation and CCR5-minus phenotypes, offering some protection against sexual acquisition of HIV-1 infection (Devito et al., 2000). However, among uninfected female commercial sex workers in Gambia, significant IgA or IgG responses in the vaginal secretions that had HIV-1 neutralizing activity were not observed (Dorrell et al., 2001). There are also reports that individuals who remain uninfected after multiple HIV exposure have CD8+ cytotoxic lymphocytes (CTLs) with strong broad-spectrum of HIV suppressive activity (Becket, 2005; Stranford et al., 1999. CD8+ CTLs have been shown to secrete ligands to CCR5, e.g., MP1α, MP1β and RANTES that can obstruct HIV entry into CD4+ cells (Cocchi et al., 1995), HIV-specific CTLs can recognize virus-derived peptides on major histocompatibility complex (MHC) Class 1 molecules and lyse the infected cells via perforin/granzyme/Fas/Fas ligand pathways (Zhou et al., 2002), and may also secrete substances that inhibit HIV replication in a nonMHC-restricted manner (Yang et al., 1997). CD8+ CTL as well as cervicovaginal mononuclear cells of HIV infected individuals are also reported to produce alpha defensin 1, 2 and 3. In individuals who do not seroconvert in spite of multiple known exposures to HIV, a robust constitutive production of α-defensin has been reported (Trabattoni et al., 2004). Different subtypes of HIV are also shown to affect both the virulence and transmissibility of HIV. Of the three main HIV subtypes: M (major), 0 (outlier) and N, the M group is reported to have at least 10 additional subtypes, that are categorized as subtypes (or clades) A through J (Kunanusont et al., 1995). Studies of discordant couples of Thailand have shown that HIV-1 subtype E may be linked to a higher risk of heterosexual transmission than subtype B, and the predominance of HIV-1 subtype E in Thailand was suggested as the cause for the rapid spread of HIV infection in Thailand (Kunanusont et al., 1995). Secretory leukocyte protease inhibitor (SLPI) is an endogenous antimicrobial molecule that inhibits the entry of HIV into CD4 and CCR5 expressing cells (Doumas et al., 2005) and an inverse relationship between HIV infection and presence of SLPI in various mucosal tissues, e.g., mouth, gut, rectum, and vagina has been reported (Naif et al., 1998). Evidence suggests that SLPI can block the internalization of HIV-1 in a dose dependent manner by inhibiting a step in viral infection that occurs after virus binding but prior to reverse transcription (McNeeley et al., 1997). Since heterosexual transmission of HIV-1 accounts for 70-80% of new HIV infection worldwide with 80% resulting from males to females (Flaskerud and Ungwarski, 1999), additional emphasis should be placed on evaluating key factors in the female genital tract that contribute to risk of HIV transmission.

With the onset of puberty, the female reproductive tract undergoes cyclic tissue destruction and remodeling every month along with the generation of an immune compromised environment. During the secretory phase of the menstrual cycle, rapid infiltration of the leukocytes takes place comprising at times, almost 40% of the total stromal cell population (Salamonsen and Lathbury, 2000). The leukocytes actively participate in endometrial tissue breakdown and remodeling, as well as in the eradication of dead sperm and bacteria, that usually accompany the semen. Their recruitment, nevertheless, is tightly controlled by hormones and cytokines, particularly during the secretory phase of the menstrual cycle, to create an immune compromised environment for embryo implantation (Kelly et al., 2001). Primary epithelial cells isolated from hysterectomized uteri express CD4, CCR5 and CXCR4 co-receptors on glandular and luminal epithelial cells (Yeaman et al., 2004). The cervix, particularly the transformation zone, is reported to be an effector site for cell mediated immunity in the female genital tract (Pundey et al., 2005). Consequently, (i) the cyclic phases of tissue breakdown and remodeling of the female reproductive tract, (ii) the availability of large numbers of CD4, CCR5 and CXCR4 co-receptors primarily at the transformation zone of the cervix, and (iii) the mandatory immune-compromised environment of the female reproductive tract each month, make the female reproductive tract vulnerable to HIV infection. In sexually active women who do not inject drugs, the genital tract is the first site that they encounter HIV. As the number of repeated tissue destruction and remodeling events increases with age, the chance for a HIV virus to gain access to the tissue persists. The findings of our study demonstrate that comparatively older women were found to have HIV protein-positive CVL samples, thereby providing additional support that these phenomena may be relevant.

The absence of PSA in CVL samples that were positive for HIV-1 p24 would argue that the male partners' semen was not the source of the p24 viral proteins (FIG. 7). Degradation of PSA was not likely since: (a) the half life of PSA is reported to be more than two days (Baum and Lipp, 2001; Johns Hopkins, 2006), (b) the presence of PSA in the study was determined by WB assays rather than being determined enzymatically, and (c) the CVL samples were all collected in tubes containing a cocktail of protease inhibitors, hence, selective degradation of PSA in CVL samples while the HIV-1 p24 protein was left intact, is improbable. Our findings suggest that some women may be infected mucosally without being infected systemically (Plicher et al., 2004), and that viral protein identified could be a marker for hitherto unrecognized HIV exposure or even subclinical infection.

An interesting finding of the present study was the presence of a 41 kDa protein in the HIV-1 p24 MAB positive CVL samples. This 41 kDa protein was seen in all the immunoblots probed with p24 monoclonal antibodies, irrespective of the clones from which the antibodies were generated (FIG. 8). The existence of a 41 kDa gag protein, a cleavage product of p55 gag protein has previously been reported (Mervis et al., 1988), which showed two types of HIV p41 gag proteins can exist: one myristoylated that resulted from the N-terminal cleavage of p55; and the other, without myristoylation, that resulted from the C-terminal processing of p55, or was synthesized de novo. The present finding of an immunopositive band at ˜41 kDa region could likewise be an HIV gag p41, or alternatively could be a dimer of p24.

Given that four of 20 women with CIN had evidence of genital tract HIV proteins, we speculate whether proteomic screening may have a place in research, to identify exposed or sub-clinically infected women, or in public health to identify women at hidden HIV risk.

Since women come to gynecology clinics for routine Pap smears, our findings demonstrate that CVL samples collected at their gynecology visits might be used to determine whether or not women are exposed to, or subclinically infected with, HIV.

### EXAMPLE 2

### Detection of HIV RNA in a CVL Sample But not the Serum Sample of a Patient Obtained at the Same Clinic Visit

Two women who were enrolled in the study described in Example 1, had also provided, with informed consent, both blood and CVL samples on the same day of their clinic visits, for HIV RNA assay by a branched DNA (bDNA) method (Bayer, Tarrytown, N.Y.). For the assay, the RNA in the specimens was first lysed and added to microtiter plates coated with specific probes, which bind the HIV RNA present in the sample. Multiple bDNA and alkaline phosphatase labeled oligonucleotide molecules were then tagged on to the immobilized HIV RNA, through a series of hybridization events. The RNA-probe complex formed is then detected using a chemiluminescent substrate; and the concentration of HIV RNA is determined from a standard curve run in parallel with the specimens. Since, a single molecule of HIV RNA may bind to 1,755 alkaline phosphatase molecules during the hybridization process; hence, the detection of the signal produced by the molecule of HIV RNA is greatly enhanced. The lower limit of sensitivity of the method used was 75 copies of HIV RNA/ml.

The bDNA assay demonstrated that the CVL samples examined had 405 copies and 2780 copies/ml of HIV RNA, respectively, whereas no HIV RNA was detected in the peripheral blood samples. If the HIV RNA in the CVL samples were to be calculated based on the amount of CVL retrieved during the CVL sampling process, the total HIV RNA retrieved in the CVL samples from the genital milieu could be calculated as 3,256 copies and 16,696 copies, respectively.

### EXAMPLE 3

### Detection of HIV-1 p24 Antigen in Biopsied Cervix Tissues of Women with HIV-Positive CVL Samples and Absence of HIV-1 p24 Antigen in Women with HIV Antigen-Negative CVL Samples

To investigate whether HIV replication was going on within the genital tract of women with HIV antigen-positive CVL samples, immunohistochemical localization of HIV p24 antigen in the cervix tissues was performed. It needs to be pointed out that the cervix biopsies used were not taken from the patients for the immunohistochemical localization study but were taken strictly for gynecologic assessment to clarify the source of their abnormal Pap smears. Formalin-fixed paraffin embedded cervix tissues left over after histopathological evaluation was used. For immunohistochemistry, 4 μm sections were cut, deparaffinized and subjected to heat-induced epitope retrieval in citrate buffer. After blocking endogenous peroxidase, the HIV-1 p24 antigen in the tissue samples was detected using monoclonal antibodies to HIV-1 p24 (Dako, Carpinteria, Calif.). Alkaline phosphatase-conjugated secondary antibody (Southern Biotech, Brimingham, Ala.) was used to visualize the antigen-antibody complex using nitroblue tetrazolium/bromo-chloro-indolyl-phosphate (NBT/BCIP) as substrate. In between each step, the sections were appropriately washed with phosphate buffered saline. Endogenous phosphatase was blocked by Levimasole. The sections were stained with either hematoxylin or Fast Red to visualize the cellular morphology. For positive controls, sections of autopsied brain tissues of an AIDS-infected patient were used; for negative controls, immunostaining of the cervix tissues were run in parallel with the primary antibody being replaced with mouse IgG, used at a concentration similar to the primary antibody.

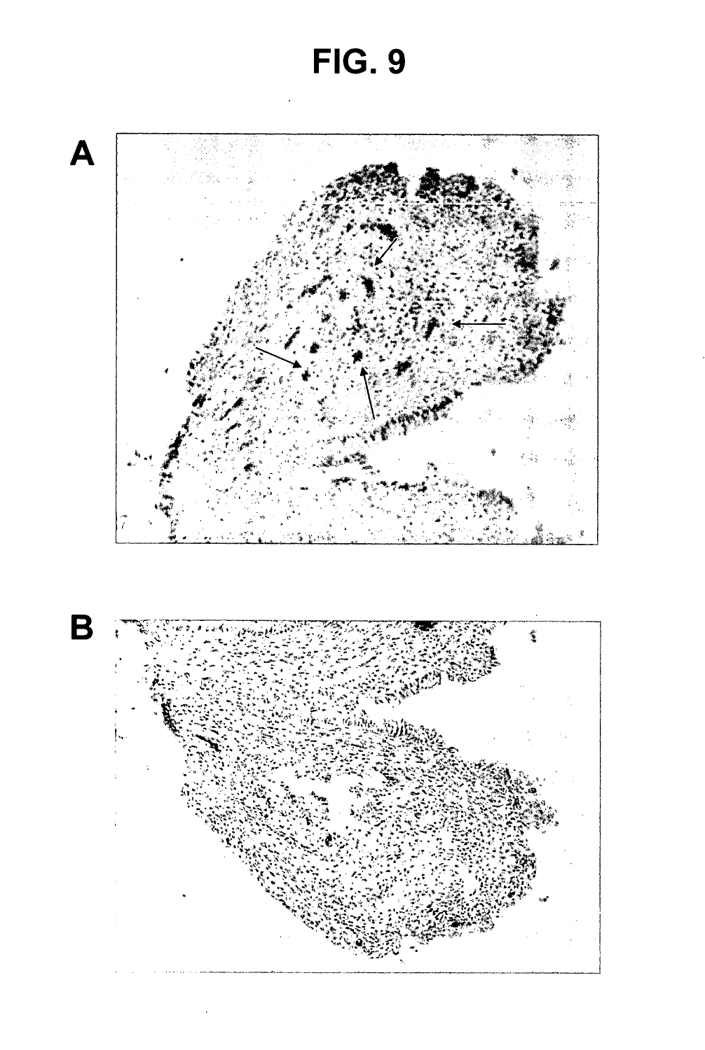

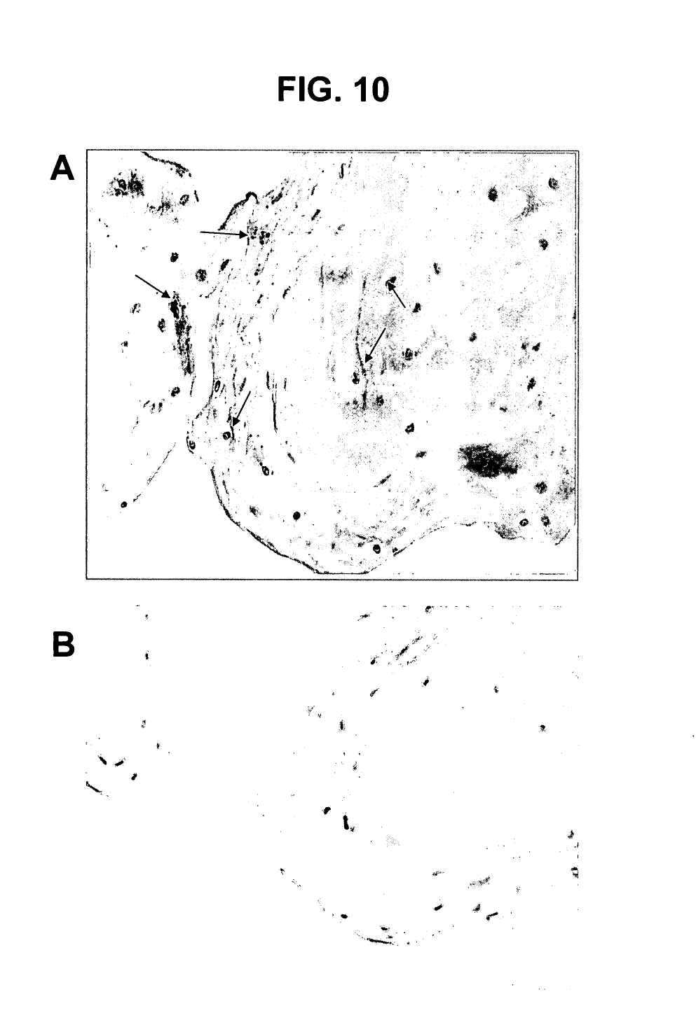

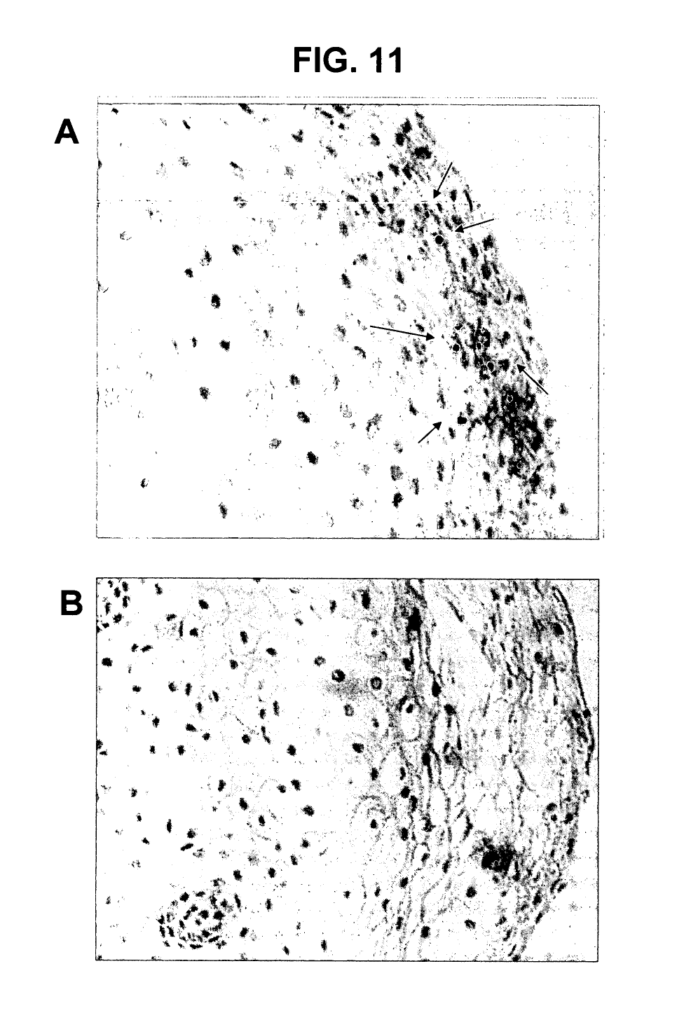

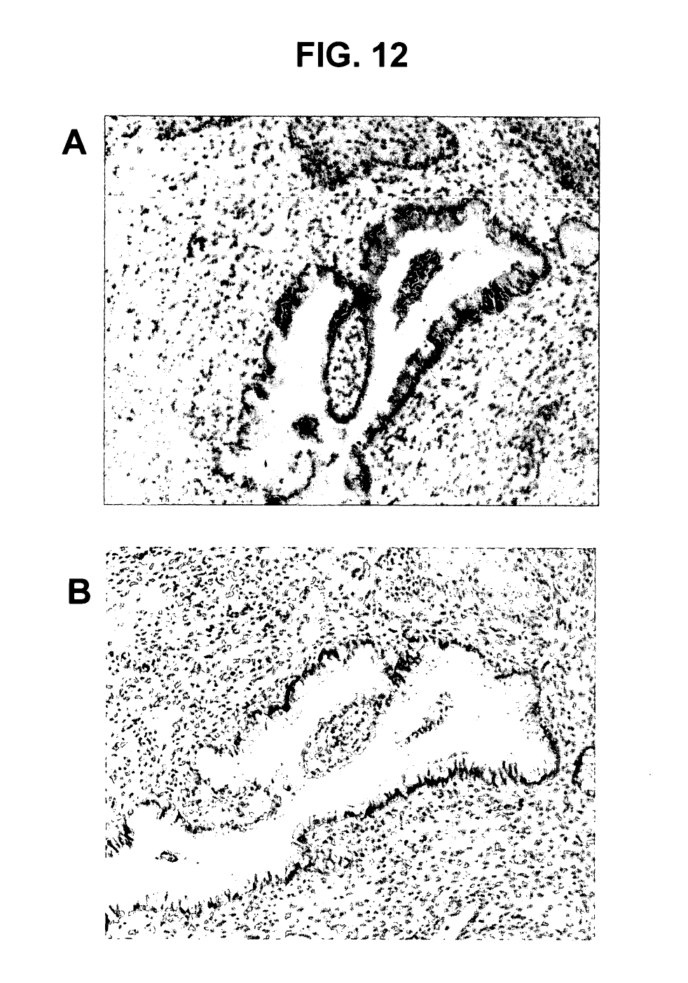

HIV-p24 antigens were localized in cervix tissues of three of our HIV-seronegative patients with HIV-positive CVL samples (FIGS. 9-11). The fourth patient with an HIV-positive CVL sample who had undergone a cone biopsy, HIV p24 antigen was not detected in the two tissue sections examined so far. It appears that all quadrants of the cone biopsy must be examined for HIV p24 antigen to conclusively determine whether the patient is HIV infected. The cervix tissues of the remaining sixteen patients in the study examined for HIV p24 antigen were found to be negative. A representative tissue section of the remaining sixteen HIV antigen-negative cervix tissues is shown in FIGS. 12A and B, treated with and without the primary antibody, respectively. The immunohistochemical results demonstrated that HIV p24 antigen was not localized throughout the tissues but were present at small loci, both in the epithelial layers of the cervix tissue as well as in the stroma. The cervix tissues of women with HIV-positive CVL samples demonstrated increased inflammation in comparison to the cervix tissues obtained from women with HIV antigen-negative CVL samples. Additionally these tissues also had elevated levels of endogenous phosphatase.

Protein tyrosine phosphatases are a large group of enzymes that regulate signal transduction processes that are critical for maintaining homeostasis and efficient cellular activation (Ouellet et al., 2003). In diseased states, specifically during HIV infection, the delicate balance between tyrosine kinase and tyrosine phosphatase is reported to be disturbed (Ouellet et al., 2003; Nekhai et al., 2007). The increased endogenous phosphatase levels in the HIV p24 antigen-positive cervix tissues in this study could be a response induced by the virus for an efficient viral replication or it could be the host's response to HIV infection to activate the resident T cells.

The characteristics of the HIV-p24 antigen-positive cells were also examined in one of the tissues (FIG. 9). The presence of HLA-DR antigen was determined to evaluate whether the HIV antigen-positive cells were macrophages or activated T cells. The results of the double staining revealed that the HIV p24 antigen-positive cells and the HLA-DR antigen positive cells were not co-localized, thereby suggesting that the HIV antigen-positive cells were neither macrophages nor T cells. Primary cells isolated from hysterectomized uteri have been shown by investigators to express CD4, CCR5 and CXCR4 co-receptors on glandular and luminal epithelial cells (Yeaman et al., 2004). The epithelial cells of HIV antigen-positive cervix tissues in this study may similarly have CD4, CCR5 and CXCR4 co-receptors expressed, allowing the virus to target these cells.

In summary, these findings conclusively show that HIV antigens, p24 and gp41 and HIV RNA can be detected in genital tract secretions and HIV p24 antigen can be detected in cervix tissues of sexually active HIV-seronegative women, substantiating that the women are not only HIV exposed but are HIV infected. It is feasible that the cohort of women identified have true mucosal HIV infection without systemic manifestations. Alternatively, these women may have aborted HIV infection with presence of some residual protein, or early HIV infection unaccompanied by a systemic antibody response.

Detection of HIV exposure in the absence of HIV antibodies in the blood was previously not known. However, the present findings demonstrate that it can now be accurately determined. These findings additionally show that CVL sampling could be an invaluable tool in screening for HIV exposure and HIV infection in asymptomatic women who are unaware of their HIV exposure or never had HIV positive results. Based on these data it is hypothesized that since women of all ages come to the gynecology clinics for routine Pap smears, techniques that are routinely employed to collect the Pap smears besides CVL sampling, e.g., the spatula, the cytobrush, the “Thin Prep” preparation etc. could all be utilized in the diagnosis of HIV exposure or subclinical infection. In the US, women in most gynecologic clinics are familiar with CVL sampling technique, hence, it is likely that they would prefer CVL sampling over standard blood testing if they are allowed to choose between the two methods. In a study in Seattle that randomized test strategies offered to clients at a needle exchange, STD clinic, and two sex clubs for men found that patients from all sites were significantly more likely to accept HIV testing when oral fluid testing was offered instead of standard blood testing (Speilberg et al., 1999). Another New York study found strong preference for oral versus venipuncture testing among prison inmates and others, when they were offered a choice between the two (Berberian, 1998).

Screening for HIV antigen(s) in the genital secretions allows individuals to be identified prior to seroconversion. If treatments are offered to these individuals at such an early stage, the treatments might be more effective in preventing the disease, particularly when their immune systems are still intact, capable of controlling viral replication. The present method of determining HIV antigen(s) in the reproductive tissues, in HIV-seronegative individuals, is a unique, clinically important and novel approach. The use of HIV antigen in genital secretions could revolutionize the way HIV screening would be implemented in the future without relying on the presence of HIV antibodies in the blood.

### REFERENCES

American Cancer Society, Cancer facts and FIGS. 2002. http://www.cancer.org/downloads/STT/CancerFacts&Figures2002™.pdf
Basu, J., C. Duttagupta, S. H. Vermund, C. Ahn, P. R. Palan, and S. L. Romney. 1993. Alterations in erythrocyte glutathione metabolism associated with cervical dysplasias and carcinoma in situ. Cancer Invest. 11:652-659.
Basu J., M. S. Mikhail, C. W. Ahn, J. Furguiele, G. Y. Ho, R. D. Burk, P. R. Palan, and S. L. Romney. 2005. Plasma uric acid levels in women with cervical intraepithelial neoplasia. Nutr. Cancer 51:25-31.
Basu J., S. L. Romney, R. H. Angeletti, E. Nieves, H. Ziao and G. A. Orr. 2005. Proteomic Analysis of Cervicovaginal Lavage Samples (CVL)L Identification of Human Immunodeficiency Virus (HIV) Proteins. Abstract 350, 2005 International Meeting of the Institute of Human Virology. Aug. 29-Sep. 3, 2005.
Baum, N., and A. Lipp. 2001. PSA for the primary care physician. Clinical Geriatrics 9:32-38.
Becker, Y. 2005. The molecular mechanism of human resistance to HIV-1 infection in persistently infected individuals-A review, hypothesis and implications. Virus Genes 31: 113-119.
Berberian E. 1998. New York state's oral fluid testing in the anonymous counseling and testing program NASTAD [National Association of State and Territorial AIDS Directors], HIV Prevention Community Bulletin, April.
Berger, E. A., P. M. Murphy, and J. M. Farber. 1999. Chemokine receptors as HIV-1 coreceptors: roles in viral entry, tropism and disease. Annu. Rev. Immunol. 17:657-700.
Bleul, C. C., L. Wu, J. A. Hoxie, T. A. Springer, and C. R. Mackay. 1997. The HIV coreceptors CXCR4 and CCR5 are differentially expressed and regulated on human T lymphocytes. Proc. Natl. Acad. Sci., USA. 94: 1925-1930.
Burk, R. D., A. S. Kadish, S. Calderin, and S. L. Romney. 1986. Human papillomavirus infection of the cervix detected by cervicovaginal lavage and molecular hybridization: correlation with biopsy results and Papanicolaou smear. Am. J. Obstet. Gynecol. 154:982-989.
Carr, S., R. Aebersold, M. Baldwin, A. Burlingame, K. Clauser, and A. Nesvizhskii. 2004. The need for guidelines in publication of peptide and protein identification data: Working group on publication guidelines for peptides and protein identification data. Mol Cell Proteomics 3:531-533.
Castle, P. E., M. Schiffman, P. E. Gravitt, H. Kendall, S. Fishman, H. Dong, A. Hildesheim, R. Herrero, M. C. Bratti, M. E. Sherman, A. Lorincz, J. E. Schussler, and R. D. Burk. 2002. Comparisons of HPV DNA detection by MY09/11 PCR methods. J. Med. Virol. 68:417-423.
Clarke B., and R. Chetty. 2002. Postmodern cancer: the role of human immunodeficiency virus in uterine cervical cancer. J. Clin. Pathol.: Mol. Pathol. 55:19-24.
Cocchi, F., A. L. DeVico, A. Garzino-Demo, S. K. Arya, R. C. Gallo, and P. Lusso. 1995. Identification of RANTES, MIP-1 alpha, and MIP-1 beta as the major HIV-suppressive factors produced by CD8+ T Cells. Science 270:1811-1815.
Devito, C., J. Hinkula, R. Kaul, L. Lopalco, J. J. Bwayo, F. Plummer, M. Cleirci, and K. Broliden. 2000. Mucosal and plasma IgA from HIV-exposed seronegative individuals neutralize a primary HIV-1 isolate. AIDS. 14:1917-1920.
Dorrell, L., A. J. Hessell, M. Wang, H. Whittle, S. Sabally, S. Rowland-Jones, D. R. Burton, and P. W. H. I. Papren. 2001. Absence of specific mucosal antibody response in HIV-exposed uninfected sex workers from the Gambia. AIDS. 14:1117-1122.
Doumas, S., A. Kolokotronis, and P. Stefanopoulos. 2005. Anti-inflammatory and antimicrobial roles of secretory leukocyte protease inhibitor. Infec. Immunity 73:1271-1274.
Dragic, T., V. Litwin, G. P. Allaway, S. R. Martin, Y. Huang, K. A. Nagashima, C. Cayanan, P. J. Maddon, R. A. Koup, J. P. Moore, and W. A. Paxton. 1996. HIV-1 entry into CD4+ cells is mediated by the chemokine receptor CC-CKR-5. Nature 381:667-673.
Flaskerud, J. H., and P. J. Ungwarski. 1999. Overview and update of HIV disease, p. 1-25. In: P. J. Ungvarski, and J. H. Flaskerud (eds.), HIV/AIDS: A guide to primary care management, W.B. Saunders Co., Philadelphia, Pa.
Irwin, K. L., C. P. Pau, D. Lupo, D. Pienazek, C. C. Lou, N. Olivo, M. Rayfield, D. J. Hu, J. T. Weber, R. A. Respess, R. Janssen, P. Minor, and J. Ernst. 1997. Presence of human immunodeficiency virus type I subtype A infection in a New York Community with high prevalence. A sentinel site for monitoring HIV genetic diversity in North America. J. Infect. Dis. 176:1629-1633.
Johns Hopkins Prostate Bulletin: Understanding PSA. http://www.phoenix5.org/Basics/UndPSAJH.html. accessed on Mar. 8, 2006.
Kedzierska, K., P. Ellery, J. Mak, S. R. Lewin, S. M. Crowe, and A. Jaworowski. 2002. HIV-1 down-modulates gamma signaling chain of Fc gamma R in human macrophages: a possible mechanism for inhibition of phagocytosis. J. Immunol. 168:2895-2903.
Kelly, R. W., A. E. King, and H. O. D. Critchley. 2001. Cytokine control in human endometrium. Reproduction 121:3-19.
Kunanusont, C., H. M. Foy, J. K. Kreiss, S. Rerks-Ngarm, P. Phanuphak, S. Raktham, C. P. Pau, and N. L. Young. 1995. HIV-1 subtypes and male to female transmission in Thailand. Lancet, London. 345:1078-1083.
Laurent, A. G., B. Krust, M. A. Rey, L. Montagnier, and A. G. Hovanessian. 1989. Cell surface expression of several species of human immunodeficiency virus type 1 major core protein. J. Virol. 63:4074-4078.
Li, L., and J. E. Dixon. 2000. Form, function, and regulation of protein tyrosine phosphatases and their involvement in human diseases. Semin. Immunol. 12:75-84.
Liu, R., W. A. Paxton, S. Choe, D. Ceradini, S. R. Martin, R. Horuk, M. E. MacDonald, H. Stuhlmann, R. A. Koup, and N. R. Landau. 1996. Homozygous defect in HIV-1 co-receptor accounts for resistance of some multiply-expose individuals to HIV-1 infection. Cell 86:367-377.

McNeeley, T. B., D. C. Shugars, M. Rosendahl, C. Tucker, S. P. Eisenberg, and S. M. Wahl. 1997. Inhibition of human immunodeficiency virus type 1 infectivity by secretory leukocyte protease inhibitor occurs prior to virus reverse transcription. Blood 90:1141-1149.
Mervis, R. J., N. Ahmad, E. P. Lillehoj, M. G. Raum, F. H. Salazar, H. W. Chan, and S. Venkatesan. 1988. The gag gene products of human immunodeficiency virus type 1: aligment within the gag open reading frame, identification of posttranslational modifications; and evidence for alternative gag precursors. J. Virol. 62:3993-4002.
Naif, H. M., S. Li, M. Alali, A. Sloane, L. Wu, M. Kelly, G. Lynch, A. Llyod, and A. L. Cunningham. 1998. CCR5 expression correlates with susceptibility of maturing monocytes to human immunodeficiency virus type 1 infection. J. Virol. 72:830-836.
Nekhai S, Jerebtsova M, Jackson A, Southerland W. 2007. Regulation of HIV-1 transcription by protein phosphatase 1. Curr. HIV Res. 5:3-9.
Nobbenhuis, M. A. E., Helmerhorst, T. J. M., van den Brule, A. J. C., Rozendaal, L., Jaspers, L. H., Voorhorst, F. J., Verheijen, R. H. M., Meijer, C. J. L. M. 2002. Primary screening for high risk HPV by home obtained cervicovaginal lavage is an alternative screening tool for unscreened women. J. Clin. Pathol. 55:435-439.
Ouellet M, Barbeau-B, Tremblay M J. 2003. Protein tyrosyl phosphatases in T cell activation: Implication for human immunodeficiency virus transcriptional activity. Prog Nucleic Acid Res. Mol. Biol. 73:69-105.
Plicher, C. D., H. C. Tien, J. J. Eron Jr., P. L. Vernazza, S. Y. Leu, P. W. Stewart, L. E. Goh, and M. S. Cohen. 2004. Brief but efficient: acute HIV infection and the sexual transmission of HIV. J. Infect. Dis. 189:1785-1792.
Phelps, R. M., D. K. Smith, C. M. Helig, L. I. Gardner, C. C. J. Carpenter, R. S. Klein, D. J. Jamieson, D. Vlahov, P. Schuman, and S. D. Holmberg. 2001. Cancer incidence in women with or at risk for HIV. Int. J. Cancer 94:753-757.
Porreco R., 1. Penn, W. Droegemueller, B. Greer, and E. Makowski. 1975. Gynecologic malignancies in immunosuppressed organ homograft receipents. Obstet. Gynecol. 45:359-364.
Pundey, J., A. J. Quayle, and D. J. Anderson. 2005. Immunological microenvironments in the human vagina and cervix: mediators of cellular immunity are concentrated in the cervical transformation zone. Biol. Reprod. 73:1253-1263.
Salamonsen, L. A., and L. I. Lathbury. 2000. Endometrial leukocytes and mensuration. Hum. Reprod. Update 6:16-27.

Samson, M., F. Libert, B. J. Doranz, J. Rucker, C. Liesnard, C. M. Farber, S. Saragosti, C. Lapoumeroulie, J. Cognaux, C. Forceille, G. Muyidermans, C. Verhofstede, G. Burtonboy, M. Georges, T. Imai, S. Rana, Y. Yi, R. J. Smyth, R. G. Coliman, R. W. Doms, G. Vassart, and M. Parmentier. 1996. Resistance to HIV-1 infection in caucasian individuals bearing mutants alleles of the CCR-5 chemokine receptor gene. Nature 382: 722-725.
Speilberg F, Goldbaum G, Branson B et al. 1999. Acceptances of alternative HIV counseling and testing strategies. Abstract 420. 1999 National HIV Prevention Conference, Atlanta, Aug. 31—Sep. 2, 1999.
Stranford, S. A., J. Skumick, D. Louria, D. Osmond, S. Chang, J. Sninsky, G. Ferrari, K. Weinhold, C. Lindquist, and J. A. Levy. 1999. Lack of infection in HIV-exposed individuals is associated with a strong CD8+ cell noncytotoxic anti-HIV response. Proc. Natl. Acad. Sci., USA. 96:1030-1035.
Suba, E. J., S. K. Murphy, A. D. Donnelly, L. M. Furia, M. L. Huynh, and S. S. Raab.
2006. Systems analysis of real-world obstacles to successful cervical cancer prevention in developing countries. Am. J. Public Health 96:480-487.
Trabattoni, D., S. L. Caputo, G. Maffeis, F. Vichi, M. Biasin, P. Pierotti, F. Fasano, M. Saressela, M. Franchini, P. Ferrante, F. Mazotta, and M. Clerici. 2004. Human [alpha] defensin in HIV-exposed but uninfected individuals. J. Acquir. Immune Defic. Syndr. 35:455-463.
Vermund S. H., M. H. Schiffman, G. L. Goldberg, D. B. Ritter, A. Weltman, and R. D. Burk. 1989. Molecular diagnosis of genital human papillomavirus infection: comparison of two methods used to collect exfoliated cervical cells. Am. J. Obstet. Gynecol. 160:304-308.
Veronese, F. D., T. D. Copeland, S. Oroszlan, R. C. Gallo, and M. G. Sarngadharan. 1988. Biochemical and immunological analysis of human immunodeficiency virus gag gene products p17 and p24. J. Virol. 62:795-801.
Yang, 0. O., S. A. Kalams, A. Trocha, H. Cao, A. Luster, R. P. Johnson, and B. D. Walker. 1997. Suppression of human immunodeficiency virus type I replication by CD8+ cells: evidence for HLA class I-restricted triggering cytolytic and noncytolytic mechanism. J. Virol. 71:3120-3128.
Yeaman, G. R., S. Asin, S. Weldon, D. J. Demain, J. E. Collins, J. L. Gonzalez, C. R. Wira, M. W. Fanger, and A. L. Howell. 2004. Chemokine receptor expression in the human ectocervix: implications for infection by the human immunodeficiency virus-type I. Immunol. 113:524-533.
Zhou, S., R. Ou, L. Huang, D. Moskophidis. 2002. Critical role for perforin-, Fas/FasL-, and TNFR1-mediated cytotoxic pathways in down-regulation of antigen-specific T cells during persistent viral infection. J. Virol. 76:829-840.

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

1: A method of diagnosing HIV in a woman, the method comprising
(a) obtaining a sample of genital tract cells and/or genital tract secretions from the woman;
(b) evaluating the sample for the presence of HIV; and
(c) determining whether the woman is HIV positive or HIV negative from the sample,
wherein the presence of HIV in the sample indicates the woman is HIV positive, and the absence of HIV in the sample indicates the woman is HIV negative.

2: The method of claim 1, wherein the sample is obtained as a Pap smear.

3: The method of claim 1, wherein the sample is obtained as a cervicovaginal lavage.

4: The method of claim 1, wherein the sample is obtained as a cytobrush.

5: The method of claim 1, wherein the sample is obtained as a Thin Prep collection.

6: The method of claim 1, wherein the sample is obtained as biopsied cervix tissue.

7: The method of claim 1, wherein the sample is obtained as endocervical curettage.

8: The method of claim 1, wherein the presence of HIV is determined by determining whether an HIV protein is present in the sample.

9: The method of claim 8, wherein the HIV protein is p24.

10: The method of claim 8, wherein the HIV protein is gp41.

11: (canceled)

12: The method of claim 8, wherein the HIV protein is detected using an antibody specific for the HIV protein.

13: The method of claim 12, wherein the antibody is a monoclonal antibody.

14: (canceled)

15: The method of claim 14, wherein the antibody was raised against an HIV protein isolated from the genital tract.

16: (canceled)

17: The method of claim 12, wherein the antibody is utilized in a western blot assay.

18-25. (canceled)

26: The method of claim 1, wherein the presence of HIV is determined by determining whether an HIV RNA is present in the sample.

27: The method of claim 26, wherein the HIV RNA is determined using reverse transcriptase-polymerase chain reaction (RT-PCR), a branched DNA assay or nucleic acid sequence-based amplification (NASBA).

28-35. (canceled)

36: The method of claim 1, wherein the HIV is HIV-1.

37: The method of claim 1, wherein the woman is HIV-seronegative.

38: A method of diagnosing HIV in an HIV-seronegative woman, the method comprising
(a) obtaining a sample of genital tract cells and/or genital tract secretions from the woman;
(b) evaluating the sample for the presence of HIV; and
(c) determining whether the woman is HIV positive or HIV negative from the sample,
wherein the presence of HIV in the sample indicates the woman is HIV positive, and the absence of HIV in the sample indicates the woman is HIV negative.

39-67. (canceled)

68: A method of determining whether to recommend that a woman should undergo anti-HIV therapy, the method comprising
(a) obtaining a sample of genital tract cells and/or genital tract secretions from the woman; and
(b) evaluating the sample for the presence of HIV,
wherein the presence of HIV in the sample indicates that anti-HIV therapy should be recommended to the woman.

69-98. (canceled)
